(19) United States
(12) Patent Application Publication
(10) Pub. No.: US 2019/0142313 A1
(43) Pub. Date: May 16, 2019

(54) SYSTEM AND METHOD FOR NON-INVASIVE CONTINUOUS REAL-TIME BLOOD GLUCOSE MONITORING

(71) Applicant: Gluco-Z GmbH, Zug (CH)

(72) Inventors: Najib Khaled Abou Ismail, Fort Drum, NY (US); Ali Mohamad AIZein, Beirut (LB); Mohammad Hassan Fadi Haroun, Beirut (LB)

(73) Assignee: Gluco-Z GmbH, Zug (CH)

(21) Appl. No.: 16/186,214

(22) Filed: Nov. 9, 2018

Related U.S. Application Data
(60) Provisional application No. 62/584,379, filed on Nov. 10, 2017.

(51) Int. Cl. A61B 5/145; A61B 5/00

(52) U.S. Cl. CPC A61B 5/14532; A61B 5/0022; A61B 5/747; A61B 5/746; A61B 5/7275; A61B 5/681

(57) ABSTRACT

A wearable blood glucose monitoring device, apparatus, and method of measuring a blood glucose level are provided. The method includes an oscillator assembly that transmits microwaves at an oscillator frequency based on an input impedance. The input impedance is associated with the permittivity of blood in a user's blood vessel. The method also includes a frequency detection circuit that detects a first oscillator frequency at a first time and a second oscillator frequency at a second time. The method further includes a main control board that receives an indication of a user's condition, compares the first oscillator frequency with the second oscillator frequency to determine a frequency drift, calibrates the frequency drift based on the received indication of the condition of the user, and determines a blood glucose level of the user based on the calibrated frequency drift. A corresponding wearable blood glucose monitoring device and apparatus are also provided.

### CROSS-REFERENCE TO RELATED APPLICATIONS

This application is claims the benefit of U.S. Provisional Application No. 62/584,379, filed Nov. 10, 2017, which application is hereby incorporated by reference in its entirety.

### TECHNOLOGICAL FIELD

The present application is directed to a system and method for a wearable device that noninvasively monitors the blood glucose level of a user. In particular, the invention is directed to a wearable device for continuous, real time in-vivo monitoring of blood glucose levels using microwaves.

### BACKGROUND OF THE INVENTION

All body parts need energy to live and work and this needed energy is produced from the food humans eat. Once the food is digested in the stomach, it is converted into glucose, or sugar, and it is immediately released into the bloodstream. In order for the body's cells to absorb and utilize the glucose to produce energy, the cells need a hormone called insulin, usually produced by the pancreas. Without insulin, glucose stays in the bloodstream and thus raises the blood's glucose level above the normal level.

Diabetes is a disease which impairs the body's ability to produce or respond to the hormone insulin, which regulates the body's blood glucose levels. It is subdivided into two types, Type 1 and Type 2. In Type 1 diabetes, the pancreas either produces very low levels of insulin or does not produce insulin at all, resulting in the diabetic individual having to inject insulin manually into his/her body. In Type 2 diabetes, either an insufficient amount of insulin is released into the blood stream or the cells becomes insulin resistant and thus cannot properly use the insulin produced by the pancreas. Type 2 diabetics have to either inject insulin or use medication that works to reduce the insulin-resistivity of the cells.

If diabetes is not diagnosed or well-treated, the glucose level in the blood gets too high and the patient may suffer from symptoms of hyperglycemia, such as extreme thirst, fatigue, dizziness, and eventually loss of consciousness. For patients who have to inject insulin manually, unplanned physical activity, unhealthy eating habits, or alcohol consumption can reduce the amount of insulin needed and can result in dangerously low levels of glucose in the patient's blood (hypoglycemia) due to the injection of too much insulin. A patient can then suffer from irregular heart rhythm, shakiness, anxiety, confusion, seizures, and eventually loss of consciousness, especially during their sleeping time, as a result of this imbalance.

These challenges make it difficult for persons with diabetes who are required to control their blood glucose levels manually, via synthetic insulin or other medication. To prevent hypoglycemia (i.e., blood glucose levels are too low, which may cause e.g. symptoms from disorientation to unconsciousness) or hyperglycemia (i.e., blood glucose levels are too high, which may cause emergency care and/or long term complications and other issues), a person with diabetes must measure their blood glucose levels on a regular basis.

### BRIEF SUMMARY

The present application is directed to systems and methods for a wearable device that noninvasively and continuously monitors the blood glucose level of a user using microwaves. The wearable device includes a resonator sensor as a passive component that is a part of an oscillator circuit, a frequency detection circuit, a main control board including an algorithm for the detection and measurement of blood glucose levels, and auxiliary sensors for compensation and calibration of glucose level estimation and prediction of hyperglycemia and hypoglycemia. The device may include a display and/or may be in wired or wireless communication with an external display device or any smart device. To continuously monitor blood glucose levels in the blood stream of the user, the oscillator, which is loaded by the resonator, radiates electromagnetic waves in the RF/microwaves range (C-band range) into the user's body, preferably at a location above an artery or any region with high vascularization under the skin.

In an example embodiment, a blood glucose monitoring device configured to be worn by a user proximate a blood vessel provided. The blood glucose monitoring device includes an oscillator assembly. The oscillator assembly includes a resonator configured to resonate at a resonator frequency based on a permittivity of blood in a user's blood vessel. The resonator is configured to provide an input impedance to the oscillator assembly. The oscillator assembly also includes an oscillator configured to transmit microwaves at an oscillator frequency based on the input impedance. The blood glucose monitoring device of an example embodiment includes a frequency detection circuit configured to detect the oscillator frequency. The blood glucose monitoring device also includes at least one auxiliary sensor configured to detect a condition of the user. The blood glucose monitoring device may further include a main control board configured to compare a first oscillator frequency detected by the frequency detection circuit at a first time with a second oscillator frequency detected by the frequency detection circuit at a second time to determine a frequency drift. The main control board may be further configured to calibrate the frequency drift based on an input received from the at least one auxiliary sensor and determine a blood glucose level of the user based on the calibrated frequency drift.

The blood glucose monitoring device of an example embodiment also includes a coating layer configured to be disposed between the oscillator assembly and the user. In such embodiments, the coating layer is configured to restrict sweat from reaching and interacting with the oscillator assembly. In an example embodiment of the blood glucose monitoring device, the resonator is a slot line resonator. In some embodiments of the blood glucose monitoring device, the oscillator is a negative resistance oscillator.

In an example embodiment of the blood glucose monitoring device, the frequency detection circuit includes at least one of a frequency discriminator, a fractional frequency divider, or a reference clock. In some embodiments of the blood glucose monitoring device, the main control board includes at least one of a frequency drift calculator, a calibration algorithm, a glucose level estimator, or a hyperglycemia/hypoglycemia predictor.

In some embodiments of the blood glucose monitoring device, the microwaves transmitted by the oscillator assembly are continuously transmitted. In various embodiments of the blood glucose monitoring device, the oscillator frequency detected in in a range from 1 gigahertz to 10 gigahertz. In some embodiments of the blood glucose monitoring device, the oscillator frequency that is detected is in a range from 4 gigahertz to 8 gigahertz.

The blood glucose monitoring device of an example embodiment also includes a user output component configured to provide an output. In such an embodiment, the output is indicative of the determined blood glucose level. In some such embodiments, the output is at least one of an audible output, a visual output, or a tactile output. In an example embodiment of the blood glucose monitoring device, the oscillator frequency is detected in a regular interval of time. In various embodiments of the blood glucose monitoring device, the condition of the user that is detected includes at least one of a sweat amount of the user, a physical activity level of the user, a sleeping time and habit of the user, or a heart rate of the user.

In another embodiment, an apparatus is provided that includes at least one processor, the at least one processor having computer-coded instructions therein, with the computer-coded instructions configured to, when executed, cause the apparatus to measure a blood glucose level. The computer program instructions are configured to, when executed, cause the apparatus to detect an oscillator frequency of microwaves transmitted by an oscillator. In such cases, the oscillator frequency is based on an input impedance associated with a permittivity of blood in a user's blood vessel. The computer program instructions are also configured to, when executed, cause the apparatus to receive an indication of a condition of the user. The computer program instructions are further configured to, when executed, cause the apparatus to compare a first oscillator frequency detected at a first time with a second oscillator frequency detected at a second time to determine a frequency drift. The computer program instructions are still further configured to, when executed, cause the apparatus to calibrate the frequency drift based on the received indication of the condition of the user. The computer program instructions are also configured to, when executed, cause the apparatus to determine a blood glucose level of the user based on the calibrated frequency drift.

In an example embodiment, the microwaves are continuously transmitted. In some embodiments, the oscillator frequency that is detected is in a range from 1 gigahertz to 10 gigahertz. In another example embodiment, the oscillator frequency that is detected is in a range from 4 gigahertz to 8 gigahertz.

In an example embodiment, the computer program instructions are configured to, when executed, cause the apparatus to provide an output. In such cases, the output is indicative of the determined blood glucose level. In some embodiments, the output is at least one of an audible output, a visual output, or a tactile output. In various embodiments, the indication of the condition of the user that is received includes an indication of at least one of a sweat amount of the user, a physical activity level of the user, a sleeping time and habit of the user, or a heart rate of the user.

In still another example embodiment, a method is provided for measuring a blood glucose level. The method includes transmitting, via an oscillator assembly, microwaves at an oscillator frequency based on an input impedance of its load. In such cases, the input impedance is associated with the permittivity of blood in a user's blood vessel. The method also includes detecting a first oscillator frequency at a first time. The method still further includes detecting a second oscillator frequency at a second time. The method also includes receiving an indication of a condition of the user. The method further includes comparing the first oscillator frequency with the second oscillator frequency to determine a frequency drift. The method still further includes calibrating the frequency drift based on the received indication of the condition of the user. The method also includes determining a blood glucose level of the user based on the calibrated frequency drift.

In an example embodiment, the microwaves at the oscillator frequency comprises continuously transmitting microwaves at the oscillator frequency. In some embodiments, the oscillator frequency that is detected is in a range from 1 gigahertz to 10 gigahertz. In various embodiments, the oscillator frequency that is detected is in a range from 4 gigahertz to 8 gigahertz.

In an example embodiment, the method also includes providing an output via a user output component, wherein the output is indicative of the determined blood glucose level. In some embodiments, the condition of the user that is detected includes at least one of an ambient temperature, a sweat amount of the user, a physical activity level of the user, a sleeping time and habit of the user, or a heart rate of the user.

The above summary is provided merely for purposes of summarizing some example embodiments to provide a basic understanding of some aspects of the invention. Accordingly, it will be appreciated that the above-described embodiments are merely examples and should not be construed to narrow the scope or spirit of the invention in any way. It will be appreciated that the scope of the invention encompasses many potential embodiments in addition to those here summarized, some of which will be further described below.

### BRIEF DESCRIPTION OF THE DRAWINGS

FIG. 1A is a block diagram of the non-invasive, wearable blood glucose monitoring device of an example embodiment of the present disclosure;

FIG. 1B illustrates a graph charting the oscillator frequency drift as it relates to the blood glucose level difference using a curve fitting model in accordance with an example embodiment of the present disclosure;

FIG. 2A is a diagram of a planar slot-line resonator used in an example embodiment of a blood glucose monitoring device in accordance with the present disclosure;

FIG. 2B illustrates an example magnetic field produced by a slot line resonator used in an example embodiment of the present disclosure;

FIG. 3 illustrates a stack view of a slot-line resonator as used in an example embodiment of the present disclosure applied to a two layered substrate (skin/artery);

FIG. 4 illustrates the resonance for an example non-superstrated slot-line resonator used in an example embodiment of the present disclosure, showing the real and imaginary parts of the input impedance (Zᵢₙ);

FIG. 5 illustrates a graph charting measured impedance of a resonant frequency at different levels of blood glucose as measured by an example embodiment of the present disclosure;

FIG. 6 illustrates a stack view of a slot-line resonator as used in an example embodiment of the present disclosure in an example configuration including a water repellent coating to account for the effects of sweat;

FIG. 7A illustrates a graph charting resonance frequency at different levels of blood glucose with sweat on a user's skin as measured by an example embodiment of the present disclosure;

FIG. 7B illustrates a graph charting measured resonance frequency of the resonator superstrated by a stacked layer including a water repellent coat, dry skin, and wet skin, fat and blood with three glucose levels as shown in the graph, in accordance with the present disclosure;

FIG. 8 is a schematic of an example negative resistance oscillator as used in an example embodiment of the present disclosure, such as a wearable blood glucose monitoring device;

FIG. 9 is a block diagram of a frequency detection circuit as used in an example embodiment of the present disclosure, such as a wearable blood glucose monitoring device;

FIG. 10 is a block diagram of the main control board as used in an example embodiment of the present disclosure, such as a wearable blood glucose monitoring device;

FIGS. 11A and 11B illustrates an exploded view of a wrist watch implementation of an example embodiment of the present disclosure, such as a wearable blood glucose monitoring device, showing the main components such as display unit, buttons, NRO-resonator PCB, water repellent coating, vibrator, coaxial cable and connectors;

FIG. 11C illustrates a compact view of a wrist watch containing an example embodiment of the present disclosure;

FIG. 11D is a side view of the wrist watch shown in FIG. 11C;

FIG. 11E is a top view of the wrist watch shown in FIG. 11C showing the display unit and main display views such as time, date, glucose level, and button functions;

FIG. 11F illustrates an exploded view of the assembled PCB and electronic components connected to the PCB in accordance with the present disclosure;

FIG. 11G illustrates the contact area of the watch with the volar part of the wrist along with the resonator and the humidity sensor mounted on the bottom strap in accordance with an example embodiment of the present disclosure;

FIG. 11H illustrate the position of the auxiliary sensors for physical activity and sleep tracking in accordance with an example embodiment of the present disclosure;

FIG. 11I illustrates the dimensions of an example wrist watch in accordance with an example embodiment of the present disclosure;

FIG. 12 is a block diagram of an apparatus configured in accordance with an example embodiment of the present disclosure; and

FIG. 13 is a flowchart illustrating the operations performed, such as by the apparatus of FIG. 12, in accordance with an example embodiment of the present disclosure.

### DETAILED DESCRIPTION

Some embodiments will now be described more fully hereinafter with reference to the accompanying drawings, in which some, but not all, embodiments are shown. Indeed, various embodiments may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present disclosure. Likewise, the terms “dielectric constant,” “relative permittivity,” and similar terms may be used interchangeably. Thus, use of any such terms should not be taken to limit the spirit and scope of embodiments of the present disclosure.

As noted above, diabetes is a serious problem for many individuals, and a person with diabetes must measure their blood glucose levels on a regular basis to guard against hypoglycemia or hyperglycemia. The earliest attempts to measure the level of glucose in a person's body focused on urine, as the glucose levels in a person's urine correspond to those in a person's blood. These measurement techniques included tablets (such as those developed by Miles Laboratories in 1941) or dipsticks that change colors, allowing an estimation of the level of glucose in a person's urine. These tests have limitations as they result in qualitative measurement only and have difficulty measuring low or normal blood glucose levels.

Further attempts focused on direct measurement of the level of glucose in blood through a patient taking a small blood sample (e.g., lancing blood from his or her finger). In 1964, Ernest Adams of Ames developed the first test strips, Dextrostix, which allowed a person to measure the level of glucose in his blood. These first test strips required manual manipulation and assessment and only produced a measurement after a sixty-second waiting period. The measurement required the user to make a visual comparison of the test strip to reference tools to identify an estimated blood glucose level. Later, electronic meters, which eliminated the need for a visual comparison and allowed for a numeric indication of blood glucose levels, were introduced. These electronic meters were not always reliable, and the detection techniques shifted to meters and testing strips that eliminated requirements that a user must wipe, blot, or wash his or her blood before testing. Measurement techniques requiring user-provided blood samples remain in wide use today and rely on electrochemical techniques to measure the level of glucose in a blood sample.

These measurement techniques requiring blood samples are limited in several ways. First, users are required to invasively extract blood samples several times per day. This traditional method is both painful and inconvenient, which can lead to non-compliance with the recommended frequency of testing. Second, the tests provide limited data—a person with diabetes has no insight as to any changes in their blood glucose between these tests. There is thus a desire for both a method of effectively measuring blood glucose in a more continuous and non-invasive manner.

Other researchers have focused on using radio frequency (RF) detector circuitry to transmit high-frequency RF waves into the body and measure properties of received signals indicative of the glucose present in blood. However, it has been shown that using this approach decreases the SNR (signal-to-noise ratio) and thus decreases the device's accuracy. Moreover, current techniques using RF measurements lack the ability to account for influencing parameters internal or external to a user's body, such as sweat, heart rate, age, gender, Diabetes Type, blood type, ambient humidity and temperature.

Further, some of these techniques use high frequency millimetric waves that cannot penetrate deep enough to target the blood vessels (such as arteries and veins) and instead target the interstitial fluid lying just underneath the skin. In this way, the glucose measurement doesn't reflect the real-time glucose level in the bloodstream, but instead reflects a measurement with a time-lag of at least 20-30 minutes.

Thus, existing devices implementing these RF measurement techniques do not produce accurate results in comparison to techniques relying on drawn blood, are not suitable for continuous monitoring, and further may not be practical for implementation on a wearable device.

There thus remains a need for a method for noninvasively measuring blood glucose in a diabetic person that can reliably and continuously monitor blood glucose levels, in the blood stream, and predict diabetes complications resulting from low and high blood glucose levels.

The present disclosure is directed to a device, an apparatus, and a method for continuously and noninvasively measuring the blood glucose level of a user. In an example embodiment, the device may include an oscillator assembly, a frequency detection circuit, and a main control board. The oscillator assembly may include an oscillator (e.g., negative resistance oscillator (NRO)) and a resonator (e.g., a slot-line resonator). In various embodiments, the oscillator may be loaded by the resonator. The device may further include auxiliary sensors, vibro-tactile alarming components, and a display. The resonator, which may be proximate to or in contact with the user's skin at an area above a blood vessel, such as an artery, or any other well vascularized area under a low-thickness skin, may be a part of the oscillator assembly.

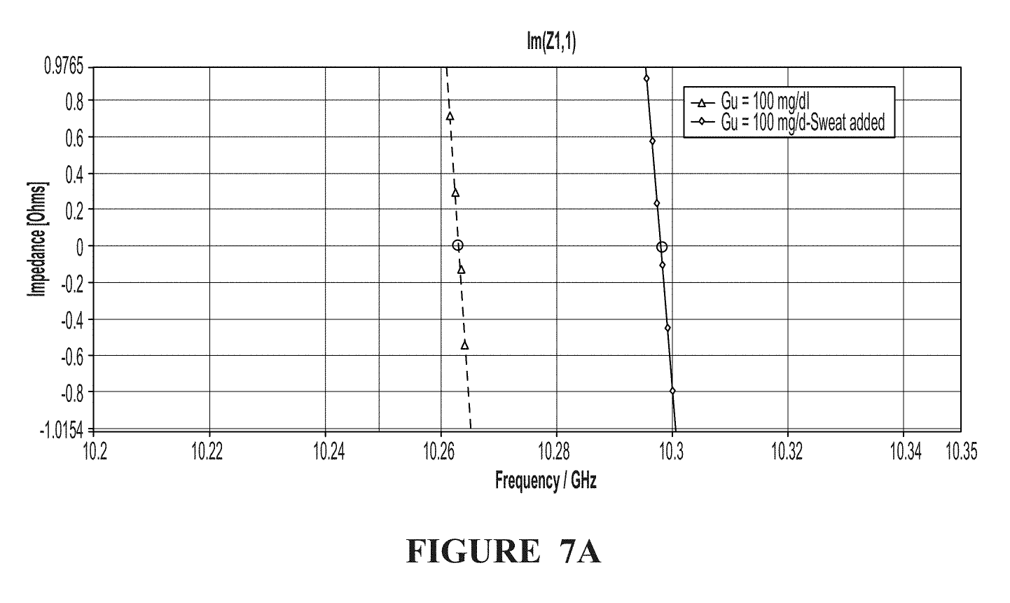

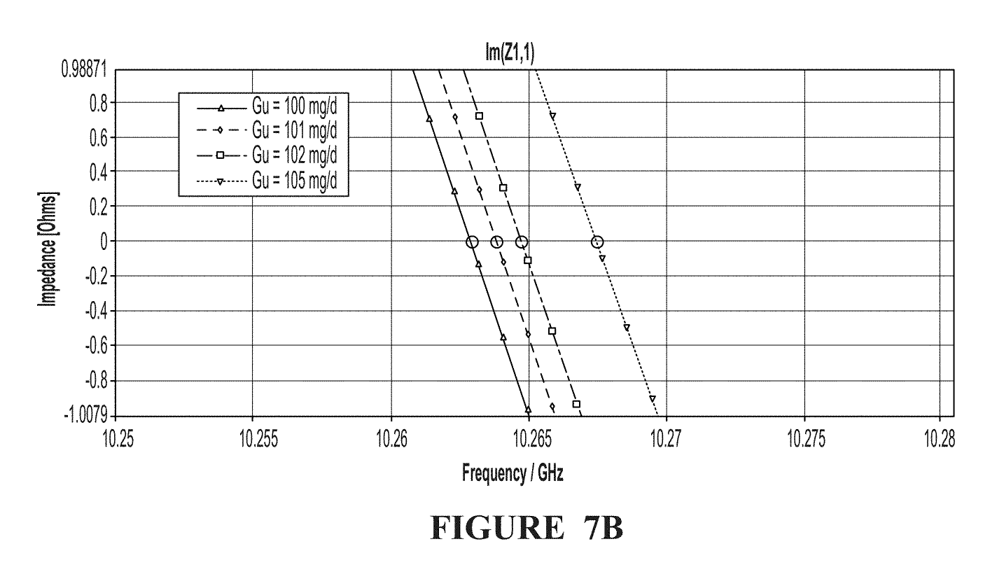

As the blood glucose levels in the user's blood stream change, the resonant frequency of the resonator will also change. The change in resonant frequency will cause the oscillator to drift its fundamental oscillation by a certain amount (e.g., there may be a shift of few MHz as shown in Table 2 corresponding to FIG. 7B), which is detected by the frequency detection circuit and transmitted to the main control board. In some embodiments, the main control board may perform a frequency drift calculation, and based on this calculated frequency drift, may determine an estimated measurement of the blood glucose levels in the user's blood. As the frequency drift is continuously sensed by the frequency detection circuit and transmitted to the main control board, the device is able to continuously monitor a user's blood glucose levels in real-time.

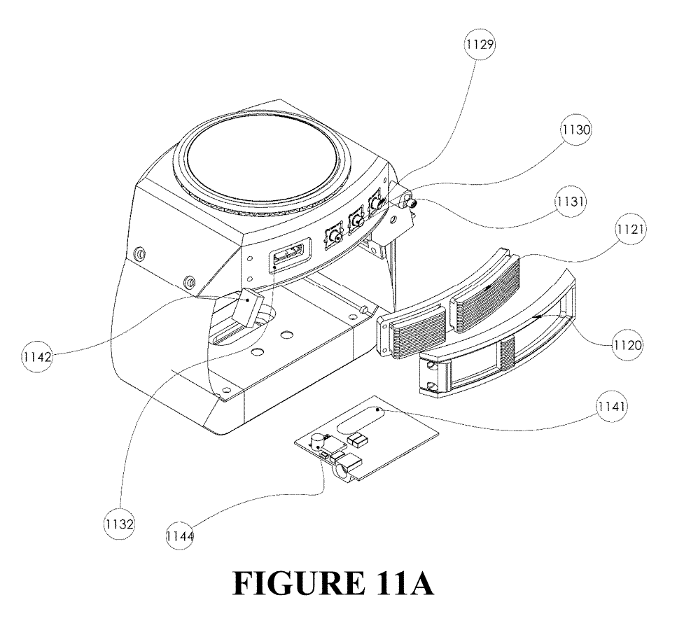

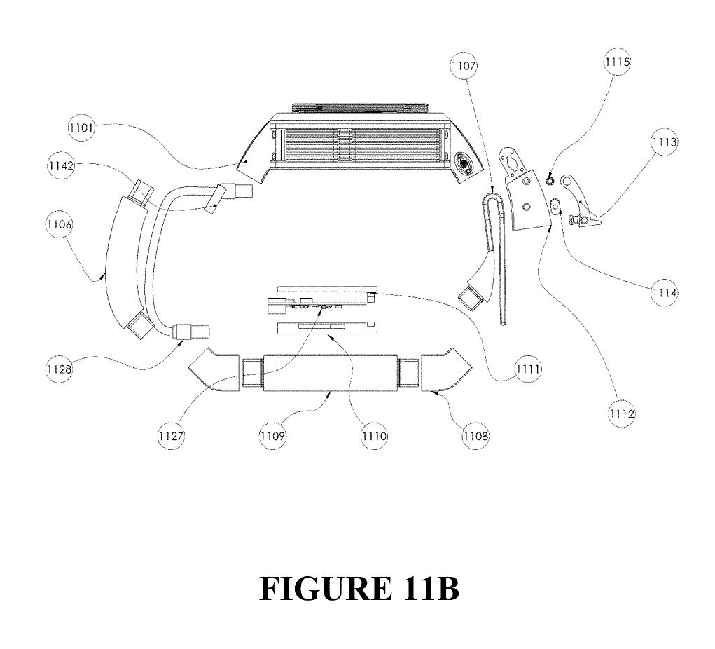

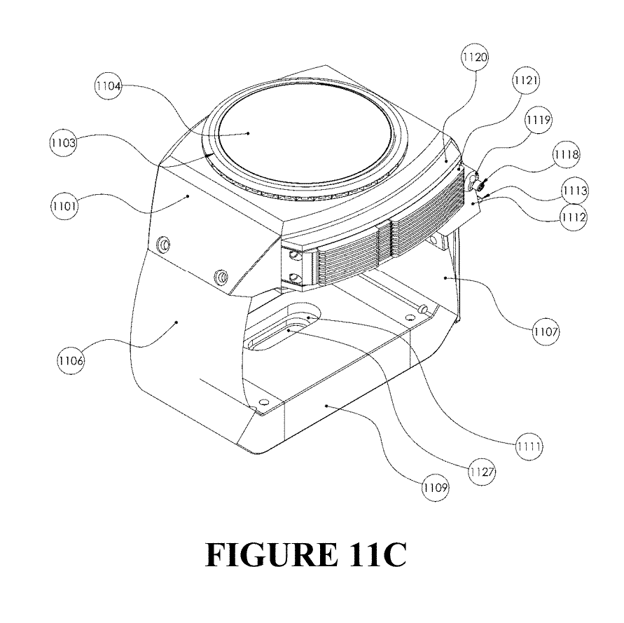

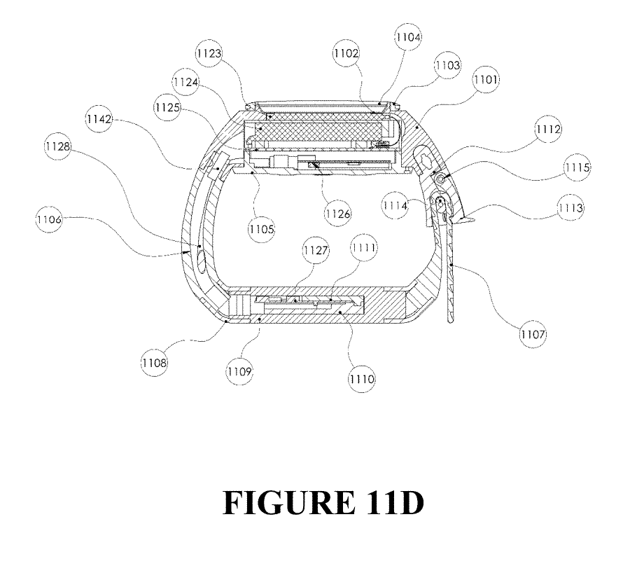

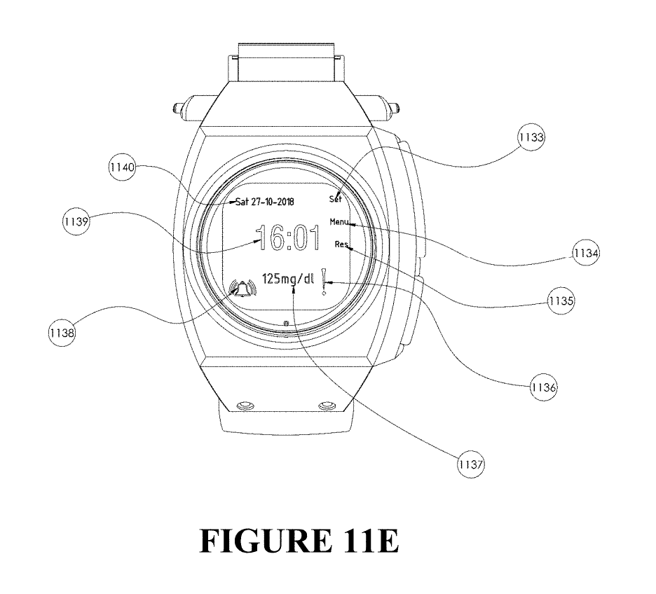

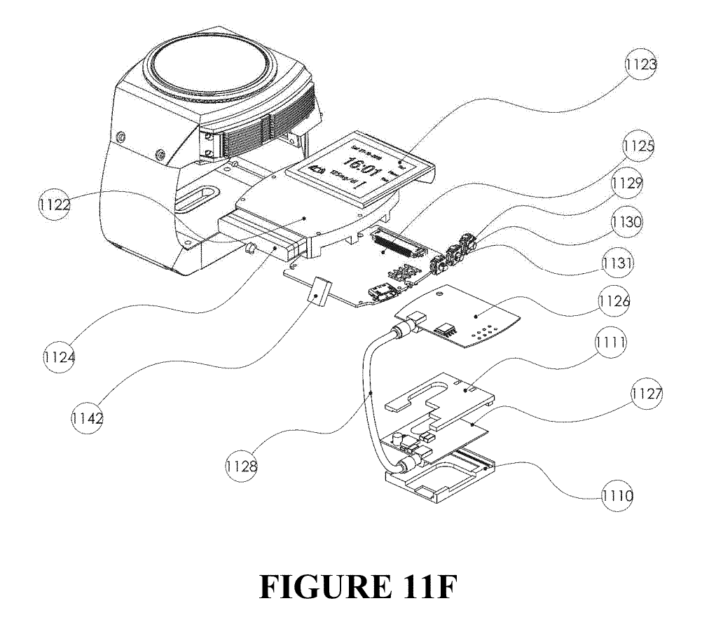

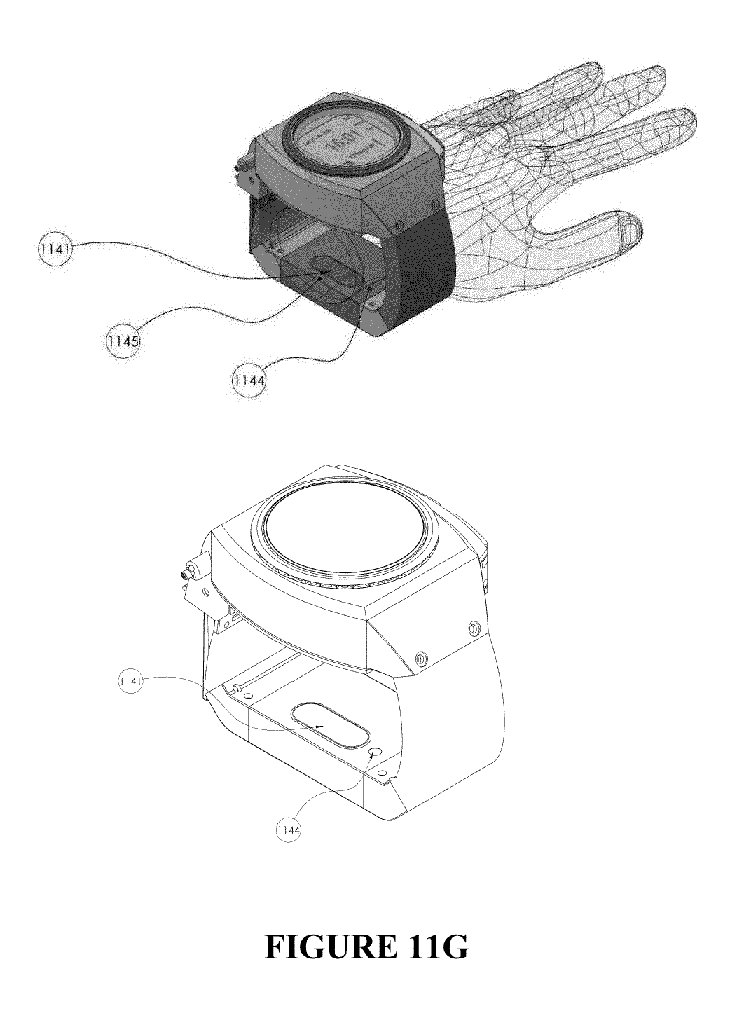

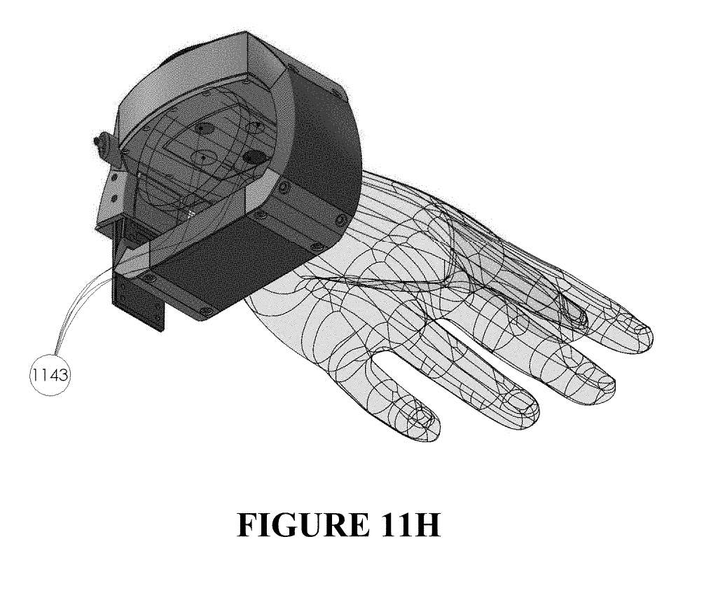

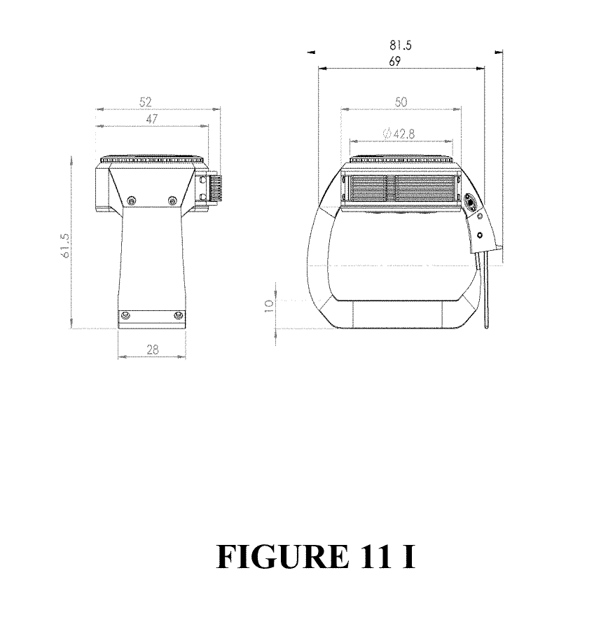

The present disclosure is aimed at creating a wearable blood glucose monitoring device, apparatus, and method that overcomes the challenges present when attempting to non-invasively measure blood glucose level. Human sweat is a conductive material, and its accumulation on a resonator may distort the physical characteristics of the resonator and prevent accurate readings. To reduce this problem, embodiments of the present invention provide for insulation of the resonator by a coating layer, such as a solder mask, a water repellent coat, and/or an insulator material. Additionally, accumulating sweat between the resonator and skin may lead to false frequency shifts. Therefore, in some embodiments, a humidity sensor may be added on the bottom strap touching the skin to detect the existence of sweat. The resonator may be mounted on the bottom strap such that it touches the skin without any air gap or space, as shown in FIG. 11G. Since each patient will have unique body parameters such as tissue thickness, metabolism, or hydration level and will be in a different environment (e.g., different ambient temperature and humidity), a calibration algorithm may be provided based on Artificial Intelligence (AI) tools such as Neural Networks and/or combined with curve fitting techniques. In order to overcome the effects of temperature and humidity variation on the dielectric constant of the resonator, a substrate material, such as R04003 or R03010, which offers a high stability of dielectric constant over temperature and humidity may be used.

An example embodiment of the present disclosure may be implemented as a wearable device. Such wearable devices may be implemented in the form of a bracelet, wrist watch, wrist band, or strap, allowing the resonator to be in direct contact with the user's skin at a location above a blood vessel, such as an artery or vein. In various embodiments, the resonator may be configured to be placed in contact with the user's wrist. In some embodiments, the casing profile of the device may also be chosen to conform to other body parts with high vascularization, such as a user's wrist or forearm, or above the user's ankle, to allow the device to have direct contact with the user's skin above such body parts.

Through wireless communication, the device may relay information to the external notification device, which would provide the user with audible and visual information and alerts regarding the monitored blood glucose levels. The device can use the features found in the connected smart device to alert the caregiver, such as SMS messaging service or GPS, to send the location of the user to the caregiver in an instance in which medical attention is required based on the determined blood sugar of the user.

Additional auxiliary sensors may be a part of or in communication with the wearable device. In various embodiments, the information from the auxiliary sensor(s) may be used for error compensation, calibration, complications prediction, and the like. Example auxiliary sensors may include humidity sensors, heart rate sensors, peripheral capillary oxygen saturation (SpO2) sensors, and 9-axis inertial motion sensors (3-axis gyroscope, 3-axis magnetometer, 3-axis accelerometer). The device may include auxiliary sensors that track user conditions such as the physical activity of the user, sleeping time, heart rate, SpO2, and humidity to detect the existence of sweat.

In an example embodiment, the watch may comprise a main compartment, display, and strap. The strap may be formed from a flexible material, such as rubber, to help maintain an appropriate grip and contact between the device and the user's skin. The main compartment may be formed with a transparent protective cover, such as an external bezel holding a transparent plastic or glass cover. A digital display, such as an LCD display, may be included beneath the transparent cover. In some embodiments, the main compartment of the wearable device may include internal molded plastic assemblies, one or more printed circuit boards, one or more processors or microprocessors 14, one or more optional sensors, rechargeable battery, alarming vibrators, and/or detection circuitry.

In an example embodiment, the resonator sensor can be superstrated directly by the wrist. The wrist may be composed of different layers of tissues, such as wet and dry skin, fat, and a blood vessel (e.g., a vein, such as a cephalic vein, a basilica vein, or any perforating vein). The input impedance (Zᵢₙ) of the load (resonator sensor+superstrate) depends on the fixed dielectric properties (relative permittivity) of the resonator+the varying dielectric properties of the tissues present above the resonator. The permittivity of the dispersive tissues changes according to changes in the constituents of each tissue. For the most part, these constituents remain relatively fixed when compared with the blood constituents in the blood stream. The variation of blood glucose level, however, is a fast changing parameter, relative to the other tissues superstrated with the resonator, and has a direct and significant effect on the overall permittivity of the superstrate of the resonator. In an example embodiment, where the permittivity is monitored, the glucose present in the blood is the main contributor to the shift of the resonant frequency of the resonator.

In other words, when the glucose level in blood varies, the permittivity of blood changes. In some embodiments, the change in permittivity of the blood affects the total overall permittivity of the wrist sensed by the resonator and causes the input impedance of the resonator to change and thus shifts the resonant frequency of the resonator. In an example embodiment, the resonant frequency is defined as the zero-crossing of the imaginary part of the input impedance (Zᵢₙ) of the resonator.

In an example embodiment, microwaves in the C-Band range of frequency (e.g., 4 to 8 gigahertz) at a power level of around −40 to 0 decibel-milliwatts (dBm) penetrate the body. Such penetration may be a few centimeters. In an example embodiment, these microwaves are produced by an electrical circuit of the oscillator assembly, where the oscillator assembly includes a negative resistance oscillator loaded by a resonator, which may be a slot line resonator. A water repellent coat may be applied on the resonator (e.g., between the device and the wearer's skin to protect the resonator from sweat or moisture present on the skin). A magnetic field may be generated by the oscillator and resonator which extends through the water repellent coat and the wearer's skin and into an artery or a vein carrying blood. As the amount of glucose in a patient's blood changes, the dielectric constant of the blood changes.

The change in the dielectric constant of the blood causes a drift in the oscillation frequency of the oscillator. As described in further detail below, a frequency detection circuit may be electrically coupled to the oscillator assembly. The frequency detection circuit may include phased locked loop (PLL) circuit with a frequency discriminator included in it. The frequency discriminator may compare the divided oscillator frequency of the oscillator and the frequency of the reference clock to produces an error signal. The error signal may then be minimized, such as by the processor 14. To minimize the error signal, the control board may vary the division factor of the oscillation frequency repeatedly until the error signal tends to zero. Once this is achieved, the control board may use the frequency of the reference clock and the last division factor to calculate the oscillation frequency of the oscillator

The main control board may also determine whether user's conditions unrelated to a change in blood glucose contribute to any change in the calculated frequency drift (e.g., accumulation of sweat layer between the resonator and the user's skin, detected by the humidity sensor integrated on the bottom strap touching the skin). These contributions may be used for calibrating the detected oscillator frequency so that it corresponds to the correct value of glucose level in blood.

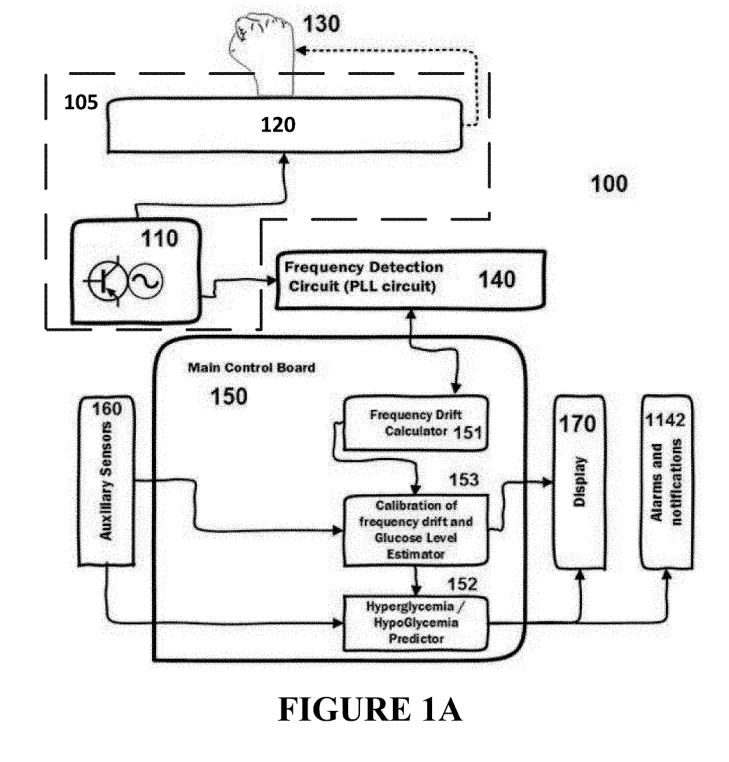

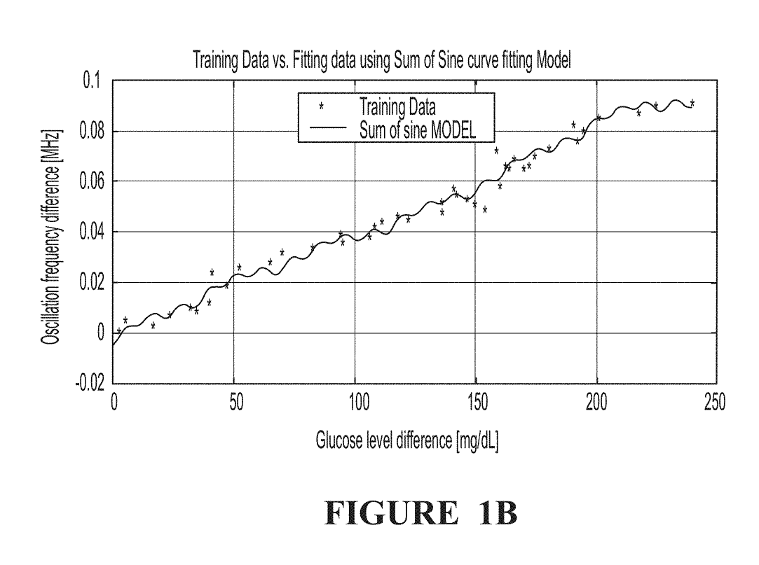

Referring now to FIG. 1A, a block diagram of an example embodiment of the present disclosure, such as a wearable blood glucose monitoring device, is provided. The wearable device may be a hand watch, wrist band, bracelet, or the like. The wearable device may include a display, for example when it is implanted as a wrist watch. Alternatively or additionally, the wearable device may include a communication interface, allowing it to communicate with external devices. This communication interface may be wired, wireless, or a combination thereof. For example, the wearable device may include near-field communication, Bluetooth, radio, Wi-Fi, or cellular components allowing it to communicate with another user device, such as a smartphone, tablet, or computer. The wearable device may also include a port for a wired connection to be made between the wearable device and external devices and may be used for charging the battery (e.g., a USB connector may be included that allows a connection to an external battery or wall port).

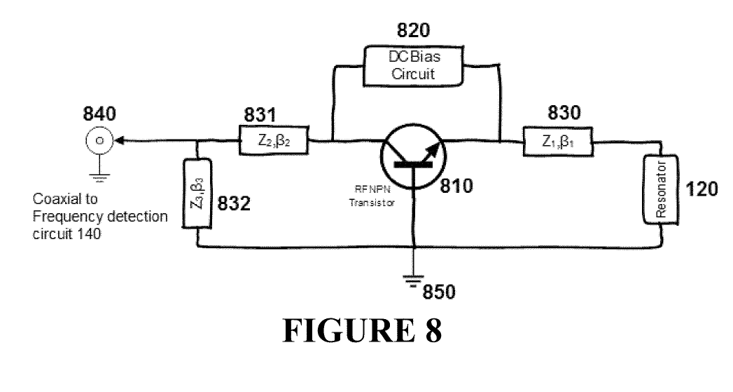

The device 100 may comprise an oscillator assembly 105. The oscillator assembly 105 may include an oscillator 110 and a resonator 120. In an example embodiment, the oscillator may be a negative resistance oscillator (NRO) 110. In some embodiments, the negative resistance oscillator may be connected to a resonator 120, such as a microwave slot line resonator. The resonator may be manufactured on the same board as the NRO or may be manufactured separately on a different board and connected to the NRO. In an example embodiment, the resonator is connected to the oscillator by a transmission line 830 (shown in FIG. 8). The resonator may be manufactured on a substrate material different than that of the NRO. The slot line resonator may rest on or near a user's skin 130, such that coupling is maintained between the resonator and a blood vessel, such as an artery or a vein, in the user's body. For example, the slot line resonator may be incorporated into a strap of the wearable device, allowing the user to tighten the strap to maintain effective coupling between the slot line resonator and the user's skin superstrate the blood vessel. In various embodiments, the oscillator may be loaded by the resonator. In such embodiments, the resonator when superstrated with a user's skin, may have a change in resonant frequency due to the change in relative permittivity of the blood. Such a change in resonant frequency will have an effect on the oscillator frequency as the resonator loads the oscillator.

The oscillator frequency is determined by the input impedance of the load connected to it (e.g., the resonator). If the device is not worn by the user, the load is the resonator alone. However, as soon as the device is worn by the user, the load of the oscillator becomes (the resonator+wrist superstrating the resonator), where the wrist is composed of all underlying tissue layers including blood which contains glucose. Therefore, when the resonator is superstrated with the user's skin, changes in the blood glucose level will result in change in the input impedance of the load which induces a drift in the oscillating frequency of the oscillator.

In various embodiments, the oscillator (e.g., NRO) may also be electrically coupled to a frequency detection circuit 140. The frequency detection circuit 140 may be configured to detect the oscillator frequency of the microwaves transmitted at a given time. In some embodiments, the frequency detection circuit may detect the oscillator frequency continuously or may detect the oscillator frequency over a certain interval of time (e.g., every 5 minutes). Additionally or alternatively, a user may place the device into a manual mode where the frequency detection circuit only detects the oscillator when the user desires.

In various embodiments, each oscillator frequency detected by the frequency detection circuit may be provided to the main control board 150. The main control board 150 may comprise a frequency drift calculator 151, a calibration algorithm 153, a glucose level estimator 153, and a hyperglycemia/hypoglycemia predictor 152. The main control board may be embodied as one or more sub control boards attached to one another through a wired connection, a wireless connection, or a combination thereof.

In certain embodiments, the frequency detection circuit may calculate the frequency drift of the oscillator according to the following equation:

Δf=fₜᵢ−fₜ₀

where fₜᵢ is the oscillator frequency at a specific measurement time and fₜ₀ is the initial oscillator frequency at the first moment the device was turned ON or put into operation.

In certain embodiments, the glucose level estimator and calibration algorithm 153, either together or separately, may use the frequency drift calculated by the frequency drift calculator 151 and a mathematical model to estimate the current value of the glucose level. The mathematical model may be pre-stored, such as in a memory device of the main control board. In some embodiments, the mathematical model may be based on an Artificial Neural Network and/or curve fitting methods in the algorithms implemented on the glucose level estimator 153 on the main control board 150.

In some embodiments, the glucose estimation algorithm 153 may relate the change in the oscillator frequency of the oscillator to the change in glucose level of the user's blood. FIG. 1B shows an example of performing such an estimation using curve fitting techniques based on Sum of Sine of order 6, where the resulting mathematical relation that is represented by the red curve is a sum of sine function multiplied by a determined coefficient or weight that may be derived from training data.

The training data (dotted in blue) is an example of an experimental data obtained for pair values (change in oscillator frequency detected by the device, change in glucose level measured invasively). The red line represents a smooth curve of a mathematical relationship or expression based on a Sum of sine of order 6 curve fitting method that estimates the relationship of the data in each pair without overfitting the data, so as to make it as generalized and global as possible.

An example General model Sin 6 function may be as follows:

f(x)=a1*sin(b1*x+c1)+a2*sin(b2*x+c2)+a3*sin(b3*x+c3)+a4*sin(b4*x+c4)+a5*sin(b5*x+c5)+a6*sin(b6*x+c6)

The distribution and sparsity of the data in this example determines the coefficients a, b, and c of the above-noted mathematical expression. For the data shown in FIG. 1B, the values of coefficients are:

| | Coefficient | | | | | | | | |
|---|---|---|---|---|---|---|---|---|---|
| | a1 | b1 | c1 | a2 | b2 | c2 | a3 | b3 | c3 |
| Value | 0.086 | 0.011 | 0.262 | 0.041 | 0.022 | 2.31 | 0.013 | 0.028 | 5.255 |

| | Coefficient | | | | | | | | |
|---|---|---|---|---|---|---|---|---|---|
| | a4 | b4 | c4 | a5 | b5 | c5 | a6 | b6 | c6 |
| Value | −0.0017 | 0.158 | 3.046 | 0.0006 | 0.397 | 3.191 | 0.0015 | 0.5536 | −1.555 |

The hyperglycemia/hypoglycemia predictor 152 may monitor the glucose level estimator and/or the auxiliary sensors to predict any expected complications that may occur for a user. In some embodiments, the monitoring by the hyperglycemia/hypoglycemia predictor may be continuous, while the device is in operation. Alternatively, the monitoring may occur at a set interval of time (e.g., every 5 minutes) or manually completed (e.g., by user input). The prediction of any expected complications may be based on a user's predefined thresholds. Additionally or alternatively, there may be predetermined thresholds of the device that would also result in a prediction of complications.

In some embodiments, the Hyper/Hypoglycemia predictor algorithm 152 may be based on a decision tree, maximum likelihood algorithms, or Naïve Bayesian network based on conditional probability calculations that can take a decision as a prediction of diabetes complications. The input parameters of this algorithm may be the current and previous glucose levels and data taken from the auxiliary sensors 160. The speed and direction of change of the estimated glucose level and the user's environmental, physical, and/or physiological conditions may help the algorithms 152 determine whether the user is going toward a hypoglycemia or hyperglycemia complication. This prediction of whether a user is experiencing, or may be about to experience, hypoglycemia or hyperglycemia may be done by a device in accordance with the present disclosure in real time.

The main control board may be connected to or in communication with auxiliary sensors 160, which may comprise sensors for measurement of heart rate, SpO2, physical activity tracking, sleep tracking, and humidity. Data from the auxiliary sensors 160 may be sent to the main control board 150, such as to the glucose level estimator, the calibration algorithm, or both, and to the hyperglycemia and hypoglycemia predictor 152. The calibration algorithm 153 may use the readings from the various auxiliary sensors to compensate for the effect of various conditions on the real frequency shift in order to prevent false detection of the user's glucose level. The calibration algorithm and the glucose level estimator may be separate or together in a component as a part of the main control board.

In some embodiments, information indicative of a user's blood glucose level, after being calculated by the glucose level estimator 153, may be passed to a user output component, such as a smart watch or mobile device. In such a case, the information, or output, may be provided to a user. In an example embodiment, the output is provided via a display 170. The information provided may include the current blood glucose level, a change in blood glucose level, a relationship to a normal blood glucose level, and/or the like. The information may be provided to the user visually, audibly, or in the form of haptic feedback. For example, there may be an alarm, discussed below, provided that alerts the user when there is a problem. In some embodiments, the display may comprise an LED or quartz display liquid crystal display, or other form of display and may also be a touchscreen. Additionally or alternatively, the device may be in communication with an external display device. For example, the main control board may be in communication with a mobile application running on an external user device, such as a smartphone or tablet. The mobile application may receive information related to the estimated blood glucose levels, desired alerts, determined blood glucose threshold level crossings, or desired displays, which it may then convey to the user.

The output may include an alert relating to the estimated blood glucose level. For example, the alert may engage when the blood glucose level may be dangerously high or low. In an example embodiment, the alert may be communicated to a user or a designated caregiver. The alerts conveyed to a user or caregiver for estimated current glucose values or predictions of complications may be based on the user's pre-defined threshold values. In some embodiments, the alerts are based on predetermined ranges of glucose values. For example, an average person may be in critical danger when a glucose level is above a certain level and therefore an alert is created if a reading is made. The designated caregiver (e.g., a family member) may be manually entered by the user (e.g., a user may enter a relative as a caregiver when they register the device), or the alert may be sent to a predetermined emergency number, such as 911. In some embodiments, the device may have an override that allows the user to instruct the device to not notify the caregiver even when the alert is triggered (e.g., the user may already be getting treated).

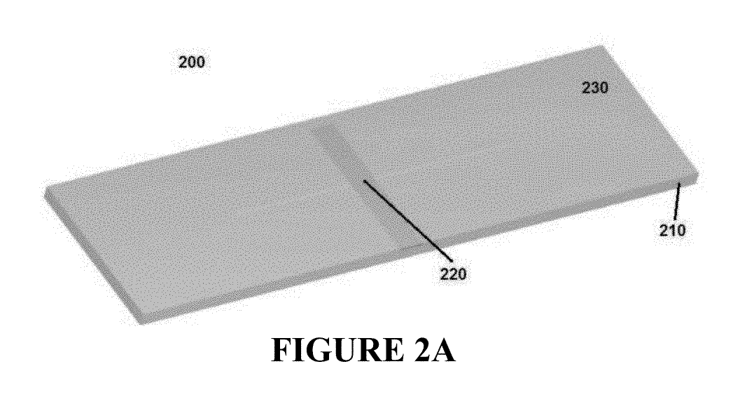

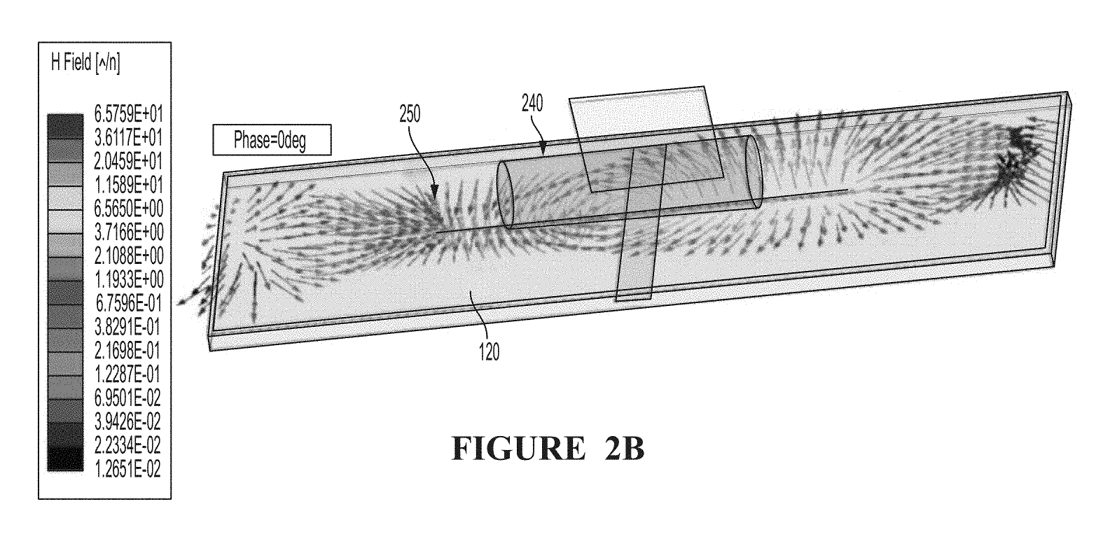

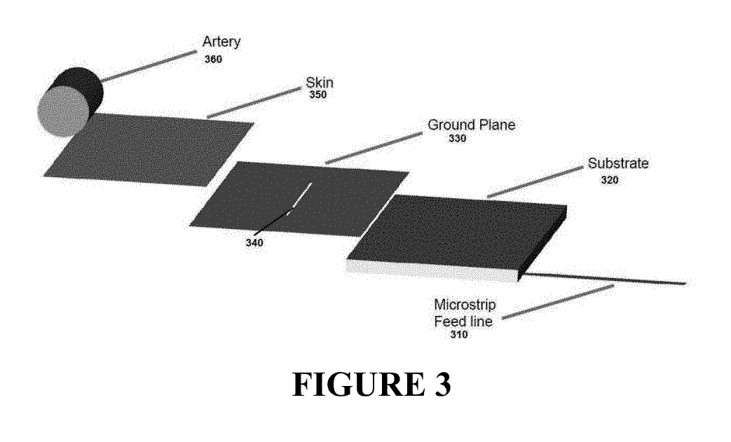

Referring now to FIG. 2A, a planar slot line resonator 200, which may be included as the resonator in an example embodiment of the present disclosure, is provided. The slot line resonator 200 may be configured to provide high magnetic coupling from the slot line to the user's exposed skin and all underlying tissue proximate the resonator. In some embodiments, the slot line resonator 200 may act as a passive load to the oscillator, such as the NRO 110 shown in FIG. 1, and its input impedance may be determined by the permittivity of the superstrate (e.g., the proximate body part). In various embodiments, the slot line resonator 200 may include a substrate 210, slot line 220, and copper ground plane 230. The substrate 210 may be covered by the copper ground plane 230, and the slot line 220 may be a double-sided shorted slot in the ground plane 230. The slot line resonator may be coupled to a feed line, which may be an etched microstrip line formed on the opposite side of the substrate, as explained in more detail below. The length of slot line resonator 200, substrate material 210, and the feed line 310 (shown in FIG. 3), in combination with the permittivity of the blood in the user's blood vessels, may determine the resonant frequency of the resonator 200.

In an example embodiment, a bracelet or watch device with the resonator integrated into a strap may require the configuration of the slot line to be changed due to size restrictions or cosmetic preferences. For example, in order to integrate the slot line resonator into the strap of the device, the slot line may be made more compact by curving or winding its structure, while keeping the same impedance properties of the straight slot line illustrated. When a user wears the device, the resonator 200, which may be configured to be susceptible to significant permittivity, may thus be positioned adjacent and parallel to the user's skin and preferably located near a blood vessel, such as an artery or a vein. Alternatively, other types of resonators that work in similar ways may be substituted for the resonator 120 of FIG. 1.

In various embodiments, the resonator 120 may project a magnetic field, as shown in FIG. 2B. The magnetic field 250 may be generated by the oscillator 110 (e.g., a NRO). The magnetic field may be in the form of microwaves. In some embodiments, the microwaves may have frequencies from 1 gigahertz to 10 gigahertz. The microwaves may be based on the resonant frequency of the resonator. As discussed above, the resonator may be positioned proximate to or in contact with an area of a person's skin that has a large amount of access to blood vessels near the skin. The microwaves may be transmitted into the blood vessel, such as an artery or vein 240, and are impeded by human tissue, including skin, muscle, fat, blood vessel, and the blood inside the blood vessel, including the glucose present in the blood. As discussed above, the relative permittivity of the blood contents affects the frequency of the resonator and, in turn, the oscillator frequency detected by the frequency detection circuit. The frequency detection circuit may, for example, detect the oscillator frequency of the oscillator and provide such frequency to the main control board. In some embodiments, the main control board may then compare the oscillator frequency received from the frequency detection circuit with a second oscillator frequency received from the frequency detection circuit at an earlier time, such as when the device is initially powered on. The main control board may then determine the blood glucose level based on the difference in the two oscillator frequencies.

Referring now to FIG. 3, a stack view of a slot line resonator of an example embodiment as implemented on the skin of a user is provided. One skilled in the art would understand that there are other ways to configure the resonator in accordance with the present disclosure. As shown in FIG. 3, a microstrip feed line 310 may be connected to the slot line resonator substrate 320. The microstrip feed line may be etched onto the bottom side of the substrate 320. This feed line may be configured to feed the slot line transmission line with electromagnetic waves. As shown, the substrate 320 may be covered by a ground plane 330, such as a copper ground plane. In some embodiments, a slot 340, which may be a double-sided shorted slot, may be included in the ground plane 330. In an example embodiment, the ground plane may be positioned adjacent to the user's skin 350, such that the slot line 340 may be superstrated to a blood vessel, such as an artery 360, under the skin 350. The slot line resonator may be connected to the oscillator using a coaxial connector (e.g., an SMP connector) and a coaxial cable as a transmission line 830 (as shown in FIG. 8).

As used in an example embodiment, the slot line resonator acts as the sensor when superstrated by the user's skin and a blood vessel. The resonant frequency of the resonator will shift to a certain frequency after contact with the skin is made. For example, a shift of 1.9 megahertz (MHz) may indicate a blood glucose level difference of 2 milligrams per deciliter (mg/dl)). In some embodiments, the oscillator frequency is related to the resonant frequency of the resonator, and may change as the dielectric constant of the blood in the blood vessel changes. As the dielectric of the blood changes, the drifts in the resonant frequency of the resonator will cause exactly the same amount of drift in the oscillator frequency. In an example embodiment, the difference between an oscillator frequency detected at a first time, such as when the device is powered on, and a second time, such as a current or instantaneous reading, may determine the frequency drift. In an example embodiment, and as discussed herein, the frequency drift may be used to determine the blood glucose level of a user.

The resonator in an example embodiment of the device may be formed using a radio frequency (RF) substrate 320. The RF substrate may be made out of a material to allow for high frequency performance. In some example embodiments, the substrate may be made out of hydrocarbon ceramic laminates, such as R04003 or R03010. In various embodiments, the substrate may be selected based on the substrate's ability to be incorporated into the strap of a watch or bracelet (e.g., practical manufacturing considerations). By tightening the strap of the watch or bracelet, appropriate coupling between the resonator and the artery may be established and maintained. As discussed above, in various embodiments the oscillator may be a negative resistance oscillator NRO. In other embodiments, the oscillator may be a voltage controlled oscillator (VCO). In various embodiments, the oscillator may be included in a compartment on the watch or bracelet. The resonator may be on the same electronic board of the oscillator, or manufactured separately and stacked on the oscillator board. For example, if the wearable device is implemented as a watch, the oscillator assembly may be incorporated into the bottom strap touching the volar part of the wrist, while the frequency detection circuit and the main control board may be placed in the central portion under the dial along with the display.

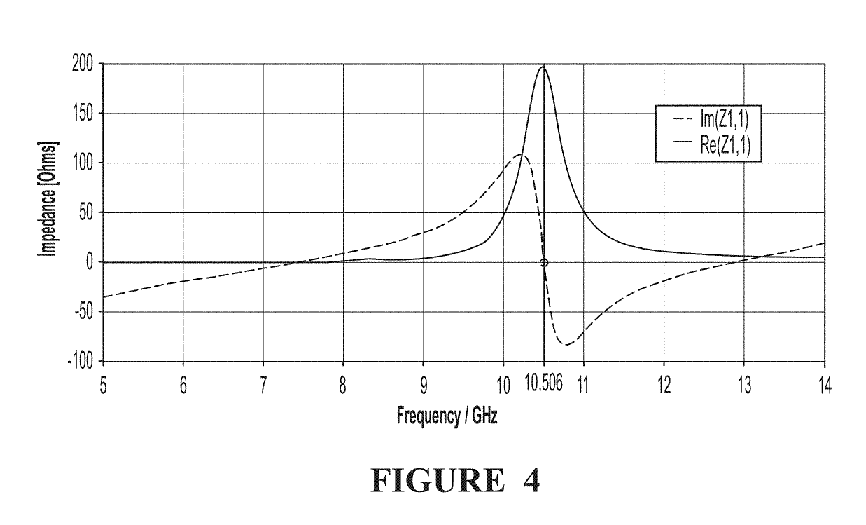

Referring now to FIG. 4, the depicted graph shows the input impedance (Zᵢₙ) of the resonator 120, as used in an example embodiment of the present disclosure when simulated alone without being superstrated to the user's skin or connected to an oscillator. In an example embodiment, the resonator, when excited by a Gaussian signal in a computer simulation, may be driven to resonate depending on the characteristics and dimensions of the resonator. The graph shown in FIG. 4 includes both the real and imaginary part of the input impedance of the resonator when not superstrated by a user's skin. The resonant frequency may be detected when the real part of the input impedance has a peak, and/or the imaginary part has a zero crossing, which is 10.506 GHz as shown in the figure. In an example embodiment, the resonator, when superstrated with a user's skin, may have a change in the resonant frequency. This change in the resonant frequency may be related to the permittivity, or dielectric constant, of the blood in a user's blood vessel (e.g., a change in the permittivity of the blood changes the impedance of the resonator). As discussed in more detail below, the frequency drift between the oscillator frequencies at two given times may be affected by the resonant frequency of the resonator at a given time. As discussed below, the resonant frequency relates directly to the oscillator frequency.

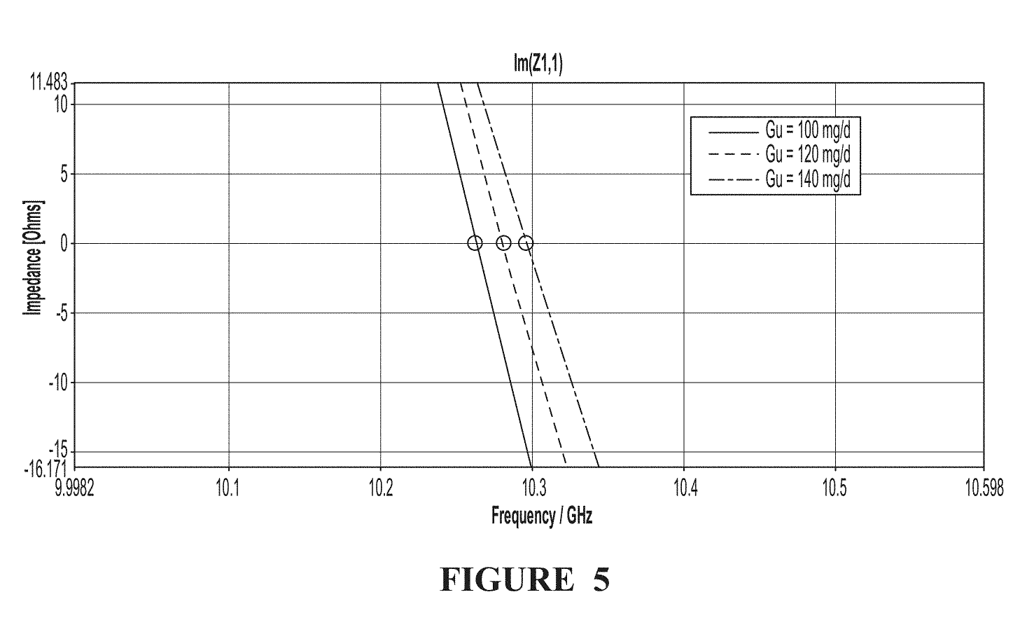

Referring now to FIG. 5, a graph shows the measured impedance at a given frequency of microwaves generated by resonator 120 of an example embodiment when superstrated to a user's skin and all underlying tissues including blood at three different glucose levels. FIG. 5 provides an illustration showing the variation of the zero crossing of the imaginary part of input impedance of the resonator as a function of glucose variation. The zero crossing of the input impedance indicates the resonant frequency. Therefore, the resonator in the figure resonates at frequencies (10.263 gigahertz (GHz), 10.281 GHz, and 10.296 GHz) for the glucose level 100 mg/dl, 120 mg/dl, and 140 mg/dl respectively. A change in blood glucose levels causes a drift in frequency. A corresponding shift in the oscillator frequency may occur which is detected by the frequency detection circuit. When the resonator 200 operates as a stand-alone component, it may resonates at a specific frequency (for example around 10 GHz). However, when the resonator 200 is connected by a transmission line 830, to the oscillator 110, the length of the transmission line 830 will lower the operating frequency of the oscillator and drive it to operate within the C-band (e.g., 4-8 GHz).

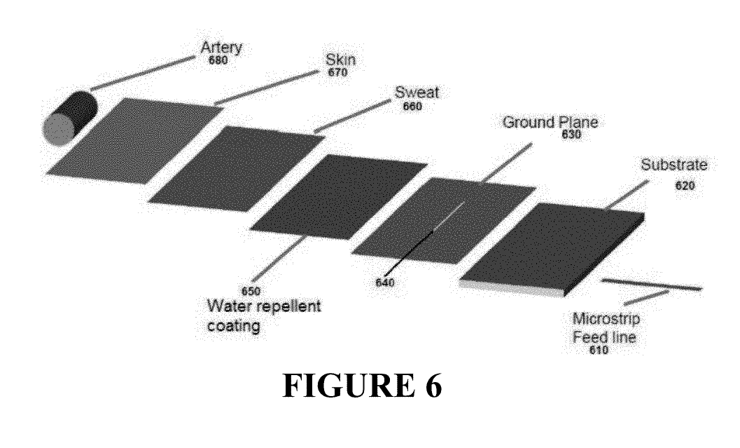

As discussed above, certain conditions may affect the accuracy and reliability of the device. In various embodiments, these conditions may include environmental conditions (e.g. temperature, humidity), physical (e.g., physical activity conditions of the user), or physiological conditions (e.g., heart rate, SpO2). For example, as discussed above, sweat or moisture may form on or near the resonator (e.g., when the user performs physical activity or in certain humid conditions). In such situations, the slot line may be in direct contact with the conductive sweat or moisture, thus degrading the performance of the oscillator and resonator. In some embodiments, to mitigate the effects of sweat or moisture accumulation on the skin of the user, a coating layer may be introduced between the resonator and the user's skin. In an example embodiment, such as the one shown in FIG. 6, the coating layer may include a water repellant coating between the resonator and the user's skin. As shown, a microstrip feed line 610, which receives an electromagnetic field from the oscillator and transmits microwave RF waves, may be connected to the bottom of the slot resonator substrate 620. The microstrip feed line 610 may be etched into the bottom surface of the substrate 620. A substrate 620 may be positioned on the ground plane 630, which includes a slot line 640.

In an example embodiment, the water-repellent coating may be made out of silicone rubber. The water-repellant coating may also be made out of other materials that have the characteristic of repelling any accumulating sweat or moisture between the resonator and the user's skin. In some embodiments, the water-repellant coat 650 may be sprayed on the ground plane, and may act to separate the resonator from sweat or moisture 660 that may form on a user's skin layer 670 in an area overtop a user's blood vessel. The water-repellant coating may restrict sweat or other moisture from reaching and interacting with the resonator. This restriction by the water-repellant coating may be in whole or in part.

An insulator material may be used (e.g., polyvinylalkohol polymes (LPA) used as a solder mask) can protect the resonator (e.g., the ground plate) from any moisture that can affect the conductivity of the material (e.g., copper). This insulator material may be used in conjunction or in place of a water repellant coating to form the coating layer. The insulator material may be of varying thickness, including thicknesses on the order of a few micrometers. As noted above, in various embodiments, the insulator material may be used in conjunction with the water-repellant coating to create the coating layer.

Referring now to FIG. 7A, a graph is provided that shows the impedance at a given frequency of microwaves generated by an oscillator 110 of FIG. 1 as a result of loading by a resonator 120 of FIG. 1, according to an example embodiment of the present disclosure, when superstrated to a user's skin with all underlying tissues including blood with a 100 mg/dl blood glucose level, and when that skin has sweat or moisture on its surface. In these tests, both lines (blue and green) are measurements according to an example embodiment of the present disclosure. This graph compares the measured resonant frequency of a resonator, such as the one shown in FIG. 3, with (blue line) and without a layer of sweat between the skin and the resonator (green line). The blue line on the graph shown in FIG. 7A is a measurement of the imaginary part of the input impedance of an example embodiment of a slot-line resonator when sweat is present, similar to the resonator shown in FIG. 3 with a layer of sweat between the ground plate 330 and the skin 350. The green line is a measure of the imaginary part of the impedance of an example embodiment of a slot-line resonator when no sweat is present, such as the resonator shown in FIG. 3. The results show that the sweat causes a major frequency shift of 34 MHz from 10.263 GHz (green line) to 10.297 GHz (blue line) at a glucose level of 100 mg/dl.

The existence of sweat or moisture may be compensated for and corrected using a calibration algorithm to ensure accurate estimation of glucose once measured through an appropriate auxiliary sensor. This calibration allows for a reduction in false alarms relating to the blood glucose level. In an example embodiment, an auxiliary sensor, such as moisture detection sensor, may be placed in the bottom strap that is in contact with user's skin. In some embodiments, additional auxiliary sensors may be used to determine additional conditions of the user. For example, one or more sensors may be provided that determine the user's physical activity level, sleeping time and habit, or heart rate.

Referring now to FIG. 7B, the measured resonant frequency of a resonator in accordance with an example embodiment superstrated by a stacked layer of, such as the one shown in FIG. 6, is provided. In this example, the stacked layers include a silicone rubber coating layer, dry skin, wet skin, fat, and a blood vessel. Four resonant frequencies of the resonator are generated when the resonator is superstrated with four different values of blood glucose levels (100, 101, 102, 105 mg/dl). In this example, the resonant frequency for 100 mg/dl, 101 mg/dl and 102 mg/dl, 105 mg/dl, respectively, were 10.2629 GHz, 10.2639 GHz, 10.2648 GHz and 10.2675 GHz. The results are shown in Table 1 and discussed in more detail below. Additionally, the corresponding oscillator frequency is also shown in Table 1. The oscillator frequency drift, as shown in table 2, corresponds to the resonator frequency drift. The oscillator frequency is generally proportional to the resonant frequency, but is lower due to transmission loss between the resonator and the oscillator, as discussed herein.

For the simulations, the slot-line resonator and its substrate were designed in order to operate around 10 GHz. The simulations were used to determine the value of the detected frequency drift produced by the resonator for four different values of glucose.

In the simulation sessions for the interaction of magnetic waves produced by the resonator and body tissues, the slot-line resonator and the superstrating layers (water repellent coat, dry skin, wet skin, fat, and blood vessel) were modeled as lossless materials. The type and thickness of the tissue layers were taken from the anatomy of the wrist. The water repellent coat and all other tissues had permittivity values that are frequency dependent according to Debye second order relaxation formula. The permittivity values of blood was frequency dependent as well as glucose dependent according to the curve fitted modified Cole-Cole model of second order. The results produced by the simulation session are shown in Tables 1 and 2 and FIG. 7B.

In the simulations, the relative permittivity value of the blood was varied to simulate a change in the glucose level in blood, where the relative permittivity of all other tissue were kept unchanged. The tissues was modeled as superstrating the resonator alone as shown in FIG. 6, and the resonator was excited by a Gaussian signal to start the simulation. In response, the input impedance of the resonator which determines its resonant frequency changes mainly due to the variation of the relative permittivity of the superstrate (e.g., the blood).

The simulation session was done on four different blood sugar levels in the range of 100-105 mg/dl, where the resonator was operating at around 10 GHz. The resonant frequency for 100 mg/dl, 101 mg/dl and 102 mg/dl, 105 mg/dl were 10.2629 GHz, 10.2639 GHz, 10.2648 GHz and 10.2675 GHz respectively.

Further, when the resonator and its load (tissue layers with varying glucose level) was put as a part of the NRO, a co-simulation session tested the oscillator frequency of the NRO for each glucose level. The oscillating frequency of the NRO for 100 mg/dl, 101 mg/dl and 102 mg/dl, 105 mg/dl were 4.6137 GHz, 4.6147 GHz, 4.6156 GHz and 4.6183 GHz respectively.

As shown, when the resonator is operating at around 10 GHz, a 5 mg/dl difference in blood glucose concentration, varying from 100 mg/dl to 105 mg/dl, results in a detected frequency drift of 4.6 MHz (10.2675-10.2629 GHz). Also, as shown in Table 1, a 1 mg/dl, 2 mg/dl, 3 mg/dl difference in blood glucose concentration results in a detected frequency drift of 1 MHz (10.2639-10.2629), 1.9 MHz (10.2648-10.2629) and 2.7 (10.2675-10.2648) MHz respectively. The graphs resulting from this simulation session is shown in FIG. 7B. We can also observe that the shifts in resonance frequency of the resonator, when simulated as standalone component, are identical to the drifts in oscillator frequency of the oscillator assembly (e.g., NRO-resonator). The results are compared in Table 1 below

TABLE 1

| | Resonance frequency of | Oscillator frequency of |
|---|---|---|
| Glucose level | the slot line resonator | the NRO |
| 100 mg/dl | 10.2629 GHz | 4.6137 GHz |
| 101 mg/dl | 10.2639 GHz | 4.6147 GHz |
| 102 mg/dl | 10.2648 GHz | 4.6156 GHz |
| 105 mg/dl | 10.2675 GHz | 4.6183 GHz |

Table 2 compares the shift in resonance frequency of the loaded resonator (tissue layers with varying glucose levels) and that of the oscillator frequency of the loaded oscillator (resonator+tissue layers with varying glucose levels).

TABLE 2

| | Resonance frequency | |
|---|---|---|
| | shift of the slot-line | Oscillator frequency |
| Difference of Glucose level | resonator | drift of the NRO |
| 5 mg/dl (100-105 mg/dl) | 4.6 MHz | 4.6 MHz |
| 3 mg/dl (102-105 mg/dl) | 2.7 MHz | 2.7 MHz |
| 2 mg/dl (100-102 mg/dl) | 1.9 MHz | 1.9 MHz |
| 1 mg/dl (100-101 mg/dl) | 1 MHz | 1 MHz |

When the resonator 200 operates as a stand-alone component, it may resonate at a specific frequency (for example around 10 GHz). However, when the resonator 200 is connected by a transmission line 830, to the oscillator 110, the length of the transmission line 830 will lower the oscillator frequency and drive it to, typically, operate within the C-band (e.g., 4-8 GHz). In some embodiments, the oscillating frequency may be in the range of 1-10 GHz.

By implementation, the minimum frequency drift detectable by the frequency detection circuit may be between 1 KHz and 50 KHz. As provided in the results shown in Table 2 and FIG. 7B, this detection resolution is more than adequate to detect a change of 1 mg/dl difference in glucose level that produces a frequency drift of 1 MHz.

Referring now to FIG. 8, a circuit schematic of a negative resistance oscillator (NRO) 110 used in an example embodiment of the present disclosure is provided. In an example embodiment, an NRO 110 may be used as the oscillator, as its oscillator frequency is varied in accordance with the change in dielectric constant of the blood. Thus, the NRO is sensitive to a change in the material properties (e.g., blood) of the superstrate of the resonator coupled to the NRO. The NRO and slot line resonator may provide magnetic coupling with the superstrate (e.g., blood vessel), allowing for accurate measurements to be obtained. In various embodiments, the negative resistance oscillator 110 may include an RF transistor 810 coupled to slot line resonator 120. In various embodiments, the entire circuit is coupled to a ground 850. In some embodiments, the transistor 810 and resonator 120 are electrically coupled to a DC Bias circuit 820. In some embodiments, the NRO may be connected through a coaxial cable to the frequency detection circuit 140. The main control board 150, described in detail with respect to FIGS. 1 and 11E herein, may be composed of the bare board which holds all the electronic components including the microprocessor. In some embodiments, the main control board may be connected to the battery and the vibrator. In various embodiments, the NRO and the resonator may be on the same circuit board connected to the frequency detection circuit 140 which is in turn connected to the main board 150. Additionally or alternatively, any portions of the oscillator assembly, the frequency detection circuit, and/or the main control board may share a circuit board and/or housing. For example, the oscillator assembly, the frequency detection circuit, and the main control board could all be connected to the same circuit board within a wearable device, such as a watch or a wrist band.

The negative resistance oscillator shown in FIG. 8 may be implemented by an RF transistor and appropriate circuitry. The resonator 120 (e.g., a slot-line resonator) may serve as a load with negative resistance, and the resonant frequency of the resonator may determine the oscillator frequency of the oscillator. In some embodiments, a change in the resonant frequency of the resonator may cause a corresponding change to the oscillator frequency of the oscillator. In some embodiments, the oscillator may be interconnected to other detection circuits by appropriate connections either wirelessly (e.g., including but not limited to near field communication (NFC) or Bluetooth™), wired (e.g., a coaxial cable), or a combination therein.

In an example embodiment, the oscillator assembly, which may include an oscillator 110 and a resonator 120, may be mounted in the strap of the watch. Once the device is worn by the user and put in operation, the oscillator 110 will start operating at an initial oscillator frequency fₜ₀. Once, the glucose level in the blood changes, the overall permittivity of the load connected to the oscillator assembly (e.g., due to the resonator and superstrated tissues and blood) will change. The change in the permittivity may cause the oscillator to shift its oscillator frequency to L. The continuously changing oscillator frequency of the oscillator will be detected by the frequency detection circuit 140.

In an example embodiment, the oscillator frequency at a given point in time may be compared to the oscillator frequency at another point in time (e.g., the initial oscillator frequency). In such a case, a relative change in the blood glucose level may be determined. The relative change in the blood glucose level may be used by the glucose level estimator 153, which may use information relating to the blood glucose level of others based on certain parameters to determine the blood glucose level. In some embodiments, this is done based on mathematical models that can deduce a change in glucose levels based on calculated oscillator frequency drifts, such as the example of curve fitting method described above in connections with FIG. 1.

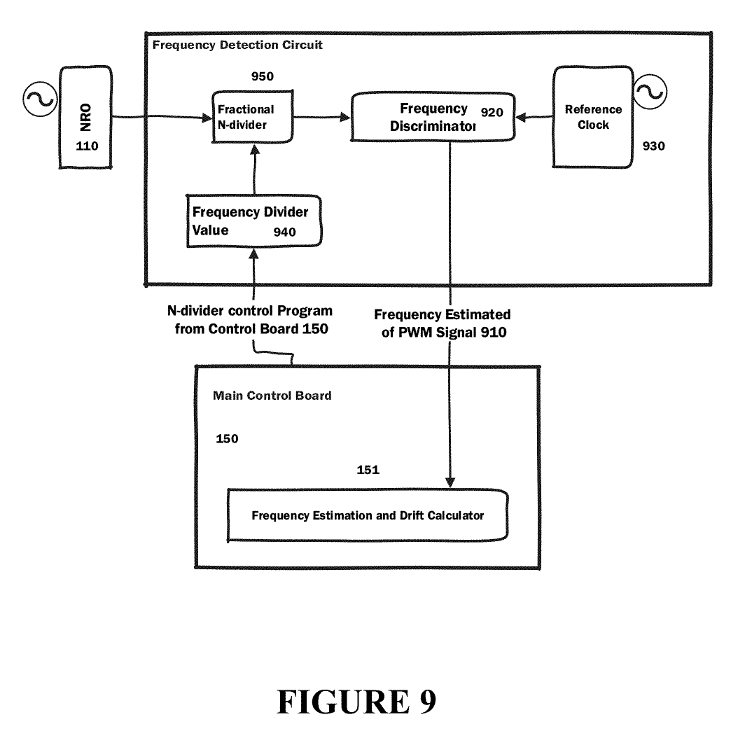

Referring now to FIG. 9, a block diagram of a frequency detection circuit 140 as used in an example embodiment of the present disclosure is provided. The oscillator 110 (e.g., an NRO) is electrically coupled to a frequency discriminator 920 based on PLL technology.

In some embodiments, a reference clock 930 may be connected to the frequency discriminator 920. For example, a reference clock 930 may be an additional oscillator, such as a Temperature Compensated Crystal Oscillator TCXO of fixed frequency within, but not restricted to, the range of 10-40 megahertz. In an example embodiment, the frequency discriminator may compare the oscillator frequency of the oscillator 110 and that of the reference clock 930. In such an embodiment, the comparison by the frequency discriminator of the oscillator frequency of the oscillator and frequency of the reference clock 930 may produce an error signal. The error signal may be in the form of a pulse width modulation (PWM) signal 910. In an example embodiment, the signal 910 is sent to a portion of the main control board 150, such as to the frequency drift calculator 151.

In various embodiments, the frequency of the PWM signal 910 of an example embodiment corresponds to the frequency difference between the oscillator frequency of the oscillator 110 and the frequency of the reference clock 930. In an example embodiment, the frequency drift may be calculated by the algorithm in the unit 151. In some embodiments, the frequency discriminator 920 may include a fractional frequency divider, such as an N-divider unit 950, which may be controlled by the main control board 150. In some embodiments, in order for the frequency discriminator to compare two input frequencies, such as the frequencies of the oscillator 110 and the reference clock 930, the frequencies need to be within the a certain range (e.g., both need to be in terms of megahertz). For example, the frequency of the oscillator 110 may be in gigahertz, while the reference clock is in megahertz. In such an example, the frequency of the oscillator may be divided by the fractional frequency divider (e.g., N-divider 950) in order for the frequency to be in megahertz. In an example case, as long as the error signal produced by the comparison of the oscillator frequency and the frequency of the reference clock is not close to zero, the main control board 150 may continue sweeping frequencies by sending a frequency divider value 940 from the fractional frequency divider to the discriminator 920 until the frequency of the PWM signal approaches zero (e.g., the oscillator frequency when combined with the appropriate frequency divider value equals the frequency of the reference clock 930), indicating that the error signal converged to zero. In an example embodiment where the error signal has converged to zero, the frequency of the reference clock when the frequency integer is incorporated is the same as the oscillator frequency.

In an example embodiment, at the moment the error signal approaches zero, the unit 151 calculates the oscillating frequency of the oscillator 110 using the frequency of the reference clock and the last frequency divider value sent by the control board 150. In various embodiments, the values of the calculated oscillator frequency of the oscillator is streamed and/or stored in the memory of the main control board 150. The stored values are then used by the unit 151 to calculate the frequency shift of the oscillator as explained in the description of FIG. 1.

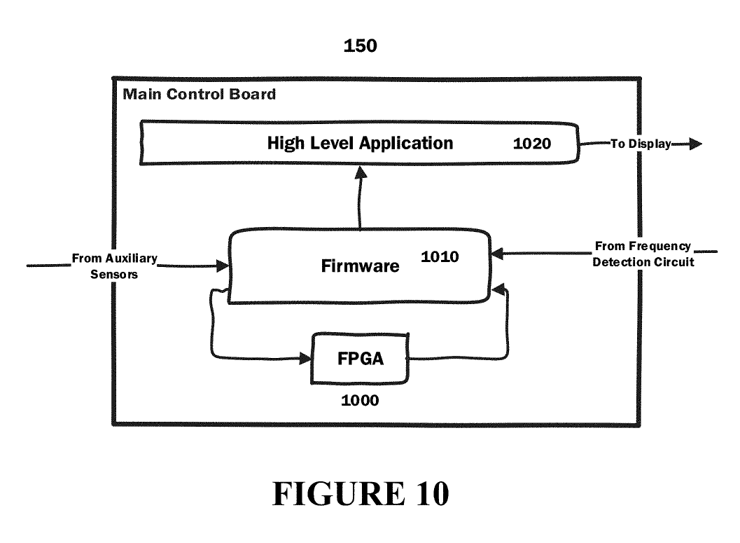

Referring now to FIG. 10, a block diagram of a main control board 150 as used in an example embodiment of the present disclosure is provided. The main control board 150 may comprise a controller 1000 which may be, but is not restricted to, a field programmable gate array (FPGA), microcontroller, or System on Chip (SOC) or other similar device. The main control board 150 may also comprise firmware 1010. Firmware 1010 may include drivers to enable the controller 1000 to interface with the frequency detection circuit 140 and the auxiliary sensors 160. The controller 1000 may use inputs from the frequency detection circuit 140 and the auxiliary sensors 160 to calculate a blood glucose level of a user. In various embodiments, the firmware 1010 also may facilitate operation of application software 1020, which may allow a user to configure, calibrate, and change settings of the device. In some embodiments, firmware 1010 may provide the blood glucose level calculated by the controller 1000 to the application software 1020. The application software 1020 may format the blood glucose level for output on display 170. Referring back to FIG. 1, an example embodiment of the main control board may be composed of 3 cores—a frequency drift calculator 151, a calibration and glucose estimation algorithm 152, and a hyper/hypoglycemia predictor algorithm 153.

In an example embodiment, at the device initialization, the calibration section 153 may perform a routine that receives initial parameters, such as humidity, heart rate, or other parameters from auxiliary sensors. In some embodiments, the calibration routine further includes determining the initial oscillator frequency of the NRO which is used as a reference frequency. After the calibration routine is performed, the wearable device may operate to monitor a user's blood glucose levels in real time, by continuously measuring and processing detected frequency drifts.

In various embodiments, the glucose level estimator, which may comprise a mathematical model based on an artificial intelligence technique or curve fitting method, takes the data from the sensors and the frequency drifts into consideration for calibration or error compensation, before estimating the glucose level. After the blood glucose level is calculated, it is compared to predetermined thresholds to determine if the user should be alerted. For example, if the calculated value is below a lower threshold or above an upper threshold, an alarm in the wearable device may be activated to alert the user. The alarm may be audible, visual, or tactile. For example, upon the calculated blood glucose level falling below a lower threshold, a vibrator in the wearable device may vibrate as an alert and the main control board may send a status to an external user device linked to the wearable device through wired or wireless communication. Further, a display on the wearable device or an external device in communication with the wearable device may show a display of the current blood glucose value along with an alert indication. In some embodiments, the main control board may send calculated blood glucose levels to an internal memory for storage, or may send information relating to the calculated blood glucose levels to an external memory, such as an external user device or server.

In an example embodiment, the glucose estimation algorithm may be in communication with a database. The database may include information taken from clinical trials, where every glucose level is related to an oscillator frequency of the oscillator. During the clinical trials, the blood glucose level of patient may be measured in vitro using conventional invasive techniques, while the oscillator frequency that corresponds to each glucose value detected by the device may be related to each measured glucose level. These clinical trials may take into account different parameters of the patient. These patient parameters may include Diabetes Type, age range, gender, blood type, etc. The user may input one or more of these patient parameters into the device (e.g., at the time he/she initiates the device for the first time). Consequently, once the initial oscillating frequency is detected, the device may automatically relate the oscillating frequency with the corresponding glucose level in the database stored, thereby determining a baseline glucose level for the user.

FIGS. 11A-11I illustrate views of an example wearable device in accordance with the present disclosure implemented as wrist watch. The present disclosure may be embodied in various ways and the discussion below should not be taken to limit the scope of the present disclosure.

FIGS. 11A and 11B illustrate an exploded view of the wrist watch, while FIG. 11C illustrates a compact view and FIG. 11D provides a side view. FIG. 11E illustrates a view of a user interface portion of the wrist watch, including user control buttons and a display. FIG. 11F illustrates an exploded view of the printed circuit board (PCB) assembly that may be used in the wrist watch. The PCB may include a main control board, a frequency detection circuit, and an oscillator assembly, as described above. FIG. 11G illustrates an example contact area between the wrist watch and the volar part of the wrist along with the resonator mounting and the humidity sensor mounted on the bottom strap. FIG. 11H shows example auxiliary sensors, such as a heart rate sensor, SpO2 sensor, and 9-axis motion detection sensor, mounted on the bottom of the upper part of the watch. FIG. 11I illustrates the dimensions of the different parts of the wrist watch.

In an example embodiment, the watch may include a protective screen or bezel 1103 overtop a watch housing 1101 that includes a display 1123. The watch housing 1101 may have one or more openings allowing for user controls, such as buttons 1129, 1130, and 1131, to interact with and connect to the watch housing 1101 and display 1123. A watch strap 1106 and 1107, which may be made of rubber, fabric, or other flexible biocompatible material, may connect to either side of the watch display housing 1101. The band straps 1106 and 1107 of the wrist watch may provide a grip of the watch on the wrist, which may be configured to establish and maintain proper grip and positioning of the resonator below the target area. This compactness may allow for more accuracy and stability of the readings.

In various embodiments, a resonator unit strap 1109 may be provided. In such embodiments, the resonator unit strap 1109 is included at a position where it would overlay the blood vessels on a user's wrist when the watch is worn. A resonator unit 1141 may be mounted on the sensor PCB 1127 (e.g., an NRO-resonator PCB) and may be covered by an insulating solder mask, such as an LPA material, and sprayed by a water repellent coating 650. The sensor unit 1127 may be sandwiched between two plastic covers 1110 and 1111 to tightly hold the PCB 1127 in place and prevent any physical interference or tampering with the electronic components on the PCB.

As shown in FIGS. 11A and 11G, one or more additional auxiliary sensors 1144, such as a humidity sensor, may be included. The auxiliary sensor(s) 1144 may be placed in the proximity of the resonator 1141 in order to accurately determine the existence of any humidity or sweat accumulating at the contact area of the resonator at the moment of the wearable device is conducting blood glucose measurement activities, allowing for a more precise calibration to account for the presence of such factors.

In some embodiments, the resonator 1141 may be formed in a recess on the watch bottom strap 1109, such that the resonator unit 1141 and insulating coating material 650 may be positioned within the recess so as to form a continuous, flat surface with the rest of the watch band, as shown in FIG. 11G.

In various embodiments, a radio frequency (RF) connection 1128, which may be a coaxial cable 1128, connects the sensor unit (oscillator assembly) 1127 to the frequency detection board 1126 in the upper case 1101 as shown in FIGS. 11B and 11F. The coaxial cable 1128 may also be positioned within a recess in the watch band 1106.

The watch band or watch housing 1101 may further include an external connector slot 1132, such as a micro USB slot. A corresponding connector port 1132 may be included in the Control board PCB 1125 that underlies the display 1123. The connector slot 1132 may allow the user to recharge the battery and/or download all or a portion of the history of the glucose levels saved over a specific period of time, such as when needed by the user or a healthcare professional. In various embodiments, other types of charging ports may be used in place of the micro USB slot to allow the user to recharge the battery and/or download all or a portion of the history of the glucose levels saved over a specific period of time.

As shown in FIG. 11E, the watch may include an interactive display 1123. In such embodiments, the display may be a touch sensitive display, an LCD screen, LED screen, OLED screen, or any other form of monitoring screen allowing the user to select options and navigate through the functions of the watch. Additionally or alternatively, the device may include user operable buttons 1129, 1130 and 1131, which connect to the display unit and allow the user to operate the device. The buttons and the USB port 1132 may be covered by a rubber pad 1121 for waterproofing, where the rubber pad is in turn held by a side cover 1120 made of the same rigid material as the case.

The display 1104 may include corresponding indicators 1133, 1134, 1135 on the display for the set, menu, and rest functions. Among other features, the display may include indicator areas for one or more of the date 1140, time 1139, current blood glucose level from the last measurement 1137, warning indicator 1136, and alarm indicator 1138. Through navigating the display, the user may be able to view past blood glucose levels, previous alerts, or previous warnings. For example, the display may show a warning symbol 1136 whenever the glucose level crosses the pre-defined maximum and minimum thresholds. In addition to the display, the crossing of these thresholds may trigger a vibrator 1142 within the wearable device to provide the user with a tactile alert. Audible warnings may also be provided through a speaker within the watch. The inclusion of a tactile alert, such as the vibrator, may be useful so that the user is alerted in situations where they cannot see the display or hear an audible alert. For example, the watch will trigger the vibrator 1142 to operate, so that it can wake up the user or alert him/her if the thresholds were crossed during sleep, exercise, or if the patient is busy or distracted. The display may also show the alarm indicator 1138 as an alarm symbol to indicate that the vibrator or audible alerts are active.

As noted, an example embodiment of the watch may include user controls, such as buttons 1129, 1130 and 1131. These buttons may connect to or make contact with the watch housing 1101 and display unit through openings in the watch housing 1101. The buttons or user controls may include a set button 1129 that may be used to choose to view the current results or request a measurement to be taken at a given time. A reset button 1131 may be included to switch off the display, switch off the alarm, or reset the measurement taken if an invasive calibration is needed. A menu button 1130 may also be included to allow the user to choose many functions that help the user view and manage his data and results. The buttons may also be used as navigation buttons to select options on the display.

In various embodiments, the functions of the wearable device may include:

A page that enables the user to enter his personal information, such as name, age, diabetes type, physician name, address, type and doses of medications if any, amount and number of insulin doses if any, and any other helpful information for the responsible physician;

An S.O.S. option that enables the user to ask for help from a caregiver (e.g., a relative or friend) in case of emergency, including automatically contacting the caregiver through the smart device connected to the watch or wrist band;

An option that enables the user to display a graph of all the glucose levels taken for a whole day or during a specified duration of time; and

An option to specify the allowable maximum and minimum thresholds of the glucose levels before an alarm can be initiated, as these thresholds may sometimes vary from patient to patient.

As shown in FIG. 11F, electronic components may be provided within the watch housing 1101, underneath the display 1123. The display unit 1123 may be held in place between an assembler 1122 and the crystal 1104. A printed circuit board assembly of the main control board 1125 connected to a battery 1124, microprocessor/controller, or micro-USB connector 1132 may be included. The assembler 1122 may have a pocket to hold the battery and help hold the display in place. In some embodiments, there may be attachment points (e.g., A-Holes) to mount the main control board PCB on its bottom face. In some embodiments, the main control board 1125 may also be connected to the underlying frequency detection circuit 1126 which is also placed in the upper part of the watch case 1101, as shown in FIG. 11F. The frequency detection circuit PCB 1126 may in turn be connected to the sensor PCB (e.g., the NRO-resonator PCB 1127) through an RF cable 1128 that passes through the cable strap 1106. The sensor PCB 1127 may be mounted in the bottom strap 1109 and sandwiched between two covers 1110 and 1111 that hold the PCB in place and avoid any physical interference or tampering of the electronic components soldered on the PCB.

In an example embodiment, the electronics may further include a memory connected to the microprocessor/controller. The memory may provide the microprocessor/controller access to data and program information that is stored in the memory and executed by the microprocessor/controller to implement the display features and control operation of the, glucose estimation and calibration unit, frequency detection circuit to detect the oscillator frequency of the oscillator, and the hyper/hypoglycemia prediction algorithm. Typically, the memory may include random access memory (RAM) circuits, read-only memory (ROM), flash memory, or a combination thereof.

The memory may also store previously calculated blood glucose levels, alerts, warnings, and user's physical activity, sleeping time or other vital parameters such as heart rate or SpO2 levels recorded by the wearable device. These auxiliary sensors 1143 may be mounted in the bottom part of the upper case 1101 as shown in FIG. 11H.

In an example embodiment, the processor may be configured to execute instructions stored in the memory device or otherwise accessible to the processor. Alternatively or additionally, the processor may be configured to execute hard coded functionality. As such, whether configured by hardware or software methods, or by a combination thereof, the processor may represent an entity (for example, physically embodied in circuitry) capable of performing operations according to an embodiment while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (for example, the computing device) configured to employ an embodiment by further configuration of the processor by instructions for performing the algorithms and/or operations described herein. The processor may include, among other things, a clock, an arithmetic logic unit (ALU) and logic gates configured to support operation of the processor.

Further, communication interfaces within the wearable watch may transmit this recorded data to external user devices, such as smartphones, tablets, or personal computers. For example, the data may be sent to a mobile device through Wi-Fi or Bluetooth communication units embedded on the circuit board. In some embodiments, a mobile application running on the mobile device may receive this data, and may provide the user with a display of information related thereto. In various embodiments, the mobile application may further provide a user interface allowing the user to adjust settings and control the wearable device. When a user selects a command or adjusts a setting in the mobile application, a signal including information regarding the user instructions may be sent to the wearable device where it is received by communication units on the circuit board. The microprocessor/controller in the wearable device may then receive these instructions, and control the wearable device accordingly.

In an example embodiment, the mobile application may further provide guidance, recommendations or instructions to the user based on the monitored blood glucose levels and alerts, such as by advising the user to consult with a physician or take an insulin shot. The mobile application may also store contact information for a user's health care providers and emergency contacts, and can include selectable instructions to automatically alert the health care providers or emergency contacts under certain conditions. For example, automatic contact may be implemented when the measured blood glucose limit falls above or below selectable thresholds, or when the number of alerts or alarms issued within a given time period is above a selected threshold.

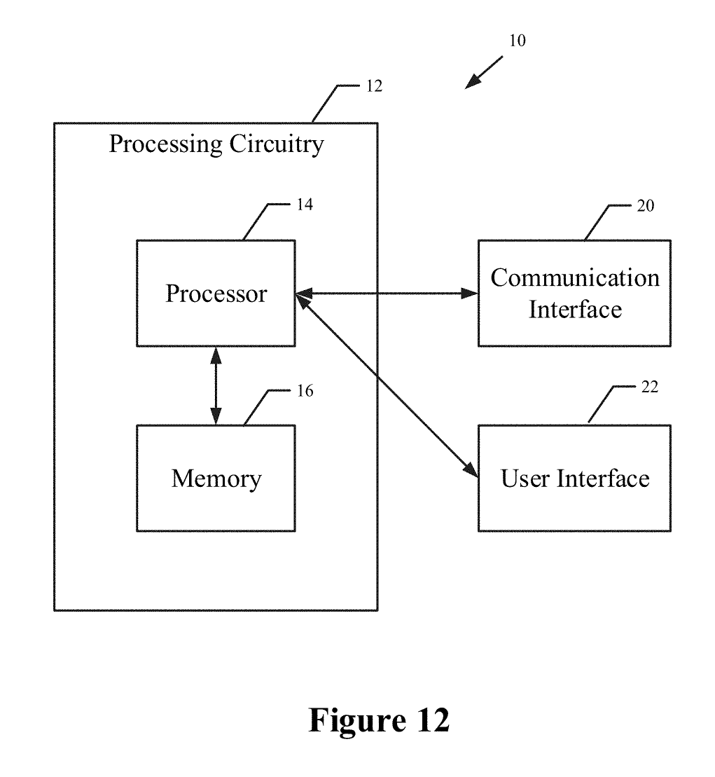

FIG. 12 is a schematic diagram of an example apparatus configured for performing any of the operations in accordance with an example embodiment as described herein. Apparatus 10 may be embodied by or associated with any of a variety of computing devices that include or are otherwise associated with a device configured for non-invasive continuous blood glucose monitoring. The apparatus may be embodied by or associated with a plurality of computing devices that are in communication with or otherwise networked with one another such that the various functions performed by the apparatus may be divided between the plurality of computing devices that operate in collaboration with one another.

The apparatus 10 may include, be associated with, or may otherwise be in communication with a processing circuitry 12, which includes a processor 14 and a memory device 16, a communication interface 20, and a user interface 22. In some embodiments, the processor 14 (and/or co-processors or any other processing circuitry assisting or otherwise associated with the processor) may be in communication with the memory device 16 via a bus for passing information among components of the apparatus. The memory device 16 may be non-transitory and may include, for example, one or more volatile and/or non-volatile memories. In other words, for example, the memory device 16 may be an electronic storage device (for example, a computer readable storage medium) comprising gates configured to store data (for example, bits) that may be retrievable by a machine (for example, a computing device like the processor). The memory device may be configured to store information, data, content, applications, instructions, or the like for enabling the apparatus to carry out various functions in accordance with an example embodiment of the present invention. For example, the memory device could be configured to buffer input data for processing by the processor. Additionally or alternatively, the memory device could be configured to store instructions for execution by the processor.

The processor 14 may be embodied in a number of different ways. For example, the processor may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor may include one or more processors configured in tandem via the bus to enable independent execution of instructions, pipelining and/or multithreading.

In an example embodiment, the processor 14 may be configured to execute instructions stored in the memory device 16 or otherwise accessible to the processor. Alternatively or additionally, the processor may be configured to execute hard coded functionality. As such, whether configured by hardware or software methods, or by a combination thereof, the processor may represent an entity (for example, physically embodied in circuitry) capable of performing operations according to an embodiment of the present invention while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (for example, the computing device) configured to employ an embodiment of the present invention by further configuration of the processor by instructions for performing the algorithms and/or operations described herein. The processor may include, among other things, a clock, an arithmetic logic unit (ALU) and logic gates configured to support operation of the processor.

The apparatus 10 of an example embodiment may also include or otherwise be in communication with a user interface 22. The user interface may include a touch screen display, a speaker, physical buttons, and/or other input/output mechanisms. In an example embodiment, the processor 14 may comprise user interface circuitry configured to control at least some functions of one or more input/output mechanisms. The processor and/or user interface circuitry comprising the processor may be configured to control one or more functions of one or more input/output mechanisms through computer program instructions (for example, software and/or firmware) stored on a memory accessible to the processor (for example, memory device 16, and/or the like). The user interface may be embodied in the same housing as the processing circuitry.

The apparatus 10 of an example embodiment may also optionally include a communication interface 20 that may be any means such as a device or circuitry embodied in either hardware or a combination of hardware and software that is configured to receive and/or transmit data from/to other electronic devices in communication with the apparatus, such as by near field communication (NFC) or other proximity-based techniques. Additionally or alternatively, the communication interface may be configured to communicate via cellular or other wireless protocols including Global System for Mobile Communications (GSM), such as but not limited to Long Term Evolution (LTE). In this regard, the communication interface may include, for example, an antenna (or multiple antennas) and supporting hardware and/or software for enabling communications with a wireless communication network. Additionally or alternatively, the communication interface may include the circuitry for interacting with the antenna(s) to cause transmission of signals via the antenna(s) or to handle receipt of signals received via the antenna(s). In some environments, the communication interface may alternatively or also support wired communication.

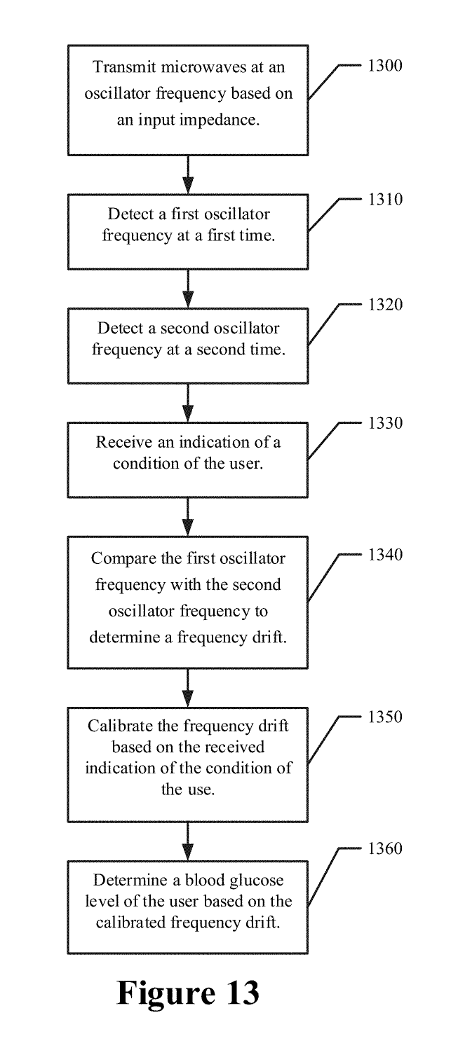

Referring now to FIG. 13, the operations performed by the apparatus 10 of an example embodiment of the present invention includes means, such as the processing circuitry 12, the processor 14 or the like, for measuring a blood glucose level. In an example embodiment, detailed above, the wearable device, apparatus, and method could be used in relation to wearable smart technology. As shown in block 1300 of FIG. 12, the apparatus 10 includes means, such as the processing circuitry 12, the processor 14 or the like, for transmitting microwaves at an oscillator frequency based on an input impedance. In an example embodiment, the transmission may be done by the oscillator assembly 105.

Referring now to block 1310 of FIG. 13, the apparatus 10 includes means, such as the processing circuitry 12, the processor 14 or the like, for detecting a first oscillator frequency at a first time. In an example embodiment, this detection may be made by the frequency detection circuit 140. Referring now to block 1320 of FIG. 13, the apparatus 10 includes means, such as the processing circuitry 12, the processor 14 or the like, for detecting a second oscillator frequency at a second time. In an example embodiment, the detection may be done by the frequency detection circuit. Referring now to block 1330 of FIG. 13, the apparatus 10 includes means, such as the processing circuitry 12, the processor 14 or the like, for receiving an indication of a condition of the user. In an example embodiment, the main control board 150 may be the recipient of the indication.

Referring now to block 1340 of FIG. 13, the apparatus 10 includes means, such as the processing circuitry 12, the processor 14 or the like, for comparing the first oscillator frequency with the second oscillator frequency to determine a frequency drift. In an example embodiment, the comparison may be completed by a portion of the main control board 150. Referring now to block 1350 of FIG. 13, the apparatus 10 includes means, such as the processing circuitry 12, the processor 14 or the like, for calibrating the frequency drift based on the received indication of the condition of the user. In an example embodiment, the calibration may be done by a portion of the main control board. Referring now to block 1360 of FIG. 13, the apparatus 10 includes means, such as the processing circuitry 12, the processor 14 or the like, for determining a blood glucose level of the user based on the calibrated frequency drift. In an example embodiment, the blood glucose level may be determined by a portion of the main control board 150.

As described above, FIG. 13 illustrates a flowchart of an apparatus 10, wearable device, and method according to example embodiments of the invention. It will be understood that each block of the flowchart, and combinations of blocks in the flowchart, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by the memory device 16 of a software development test platform employing an embodiment of the present invention and executed by the processing circuitry 12, the processor 14 or the like of the software development test platform. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowchart support combinations of means for performing the specified functions and combinations of operations for performing the specified functions for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

In some embodiments, certain ones of the operations above may be modified or further amplified. Furthermore, in some embodiments, additional optional operations may be included. Modifications, additions, or amplifications to the operations above may be performed in any order and in any combination.

The foregoing disclosure and description of the disclosed embodiments is illustrative and explanatory of the embodiments of the invention. Various changes in the details of the illustrated embodiments can be made within the scope of the appended claims without departing from the true spirit of the disclosure. The embodiments of the present disclosure should only be limited by the following claims and their legal equivalents.

That which is claimed:

1. A blood glucose monitoring device configured to be worn by a user proximate a blood vessel, the device comprising:
an oscillator assembly comprising a resonator configured to resonate at a resonator frequency based on a permittivity of blood in a user's blood vessel, wherein the resonator is configured to provide an input impedance to the oscillator assembly, wherein the oscillator assembly further comprises an oscillator configured to transmit microwaves at an oscillator frequency based on the input impedance;
a frequency detection circuit configured to detect the oscillator frequency;
at least one auxiliary sensor configured to detect a condition of the user; and
a main control board configured to compare a first oscillator frequency detected by the frequency detection circuit at a first time with a second oscillator frequency detected by the frequency detection circuit at a second time to determine a frequency drift, the main control board being further configured to calibrate the frequency drift based on an input received from the at least one auxiliary sensor,
wherein the main control board is configured to determine a blood glucose level of the user based on the calibrated frequency drift.

2. The device of claim 1 further comprising a coating layer configured to be disposed between the oscillator assembly and the user, wherein the coating layer is configured to restrict sweat from reaching and interacting with the oscillator assembly.

3. The device of claim 1, wherein the resonator is a slot line resonator.

4. The device of claim 1, wherein the oscillator is a negative resistance oscillator.

5. The device of claim 1, wherein the frequency detection circuit includes at least one of a frequency discriminator, a fractional frequency divider, or a reference clock.

6. The device of claim 1, wherein the main control board includes at least one of a frequency drift calculator, a calibration algorithm, a glucose level estimator, or a hyperglycemia/hypoglycemia predictor.

7. The device of claim 1, wherein the microwaves transmitted by the oscillator assembly are continuously transmitted.

8. The device of claim 1, wherein the oscillator frequency that is detected is in a range from 1 gigahertz to 10 gigahertz.

9. The device of claim 1, wherein the oscillator frequency that is detected is in a range from 4 gigahertz to 8 gigahertz.

10. The device of claim 1 further comprising a user output component configured to provide an output, wherein the output is indicative of the determined blood glucose level.

11. The device of claim 10, wherein the output is at least one of an audible output, a visual output, or a tactile output.

12. The device of claim 1, wherein the oscillator frequency is detected in a regular interval of time.

13. The device of claim 1, wherein the condition of the user that is detected includes at least one of a sweat amount of the user, a physical activity level of the user, a sleeping time and habit of the user, or a heart rate of the user.

14. An apparatus for measuring a blood glucose level comprising at least one processor, the at least one processor having computer-coded instructions therein, with the computer-coded instructions configured to, when executed, cause the apparatus to:
detect an oscillator frequency of microwaves transmitted by an oscillator, wherein the oscillator frequency is based on an input impedance associated with a permittivity of blood in a user's blood vessel;
receive an indication of a condition of the user;
compare a first oscillator frequency detected at a first time with a second oscillator frequency detected at a second time to determine a frequency drift;
calibrate the frequency drift based on the received indication of the condition of the user; and
determine a blood glucose level of the user based on the calibrated frequency drift.

15. The apparatus of claim 14, wherein the microwaves are continuously transmitted.

16. The apparatus of claim 14, wherein the oscillator frequency that is detected is in a range from 1 gigahertz to 10 gigahertz.

17. The apparatus of claim 14, wherein the oscillator frequency that is detected is in a range from 4 gigahertz to 8 gigahertz.

18. The apparatus of claim 14 further comprising computer-coded instructions configured to, when executed, cause the apparatus to provide an output, wherein the output is indicative of the determined blood glucose level.

19. The apparatus of claim 18, wherein the output is at least one of an audible output, a visual output, or a tactile output.

20. The apparatus of claim 14, wherein the indication of the condition of the user that is received includes an indication of at least one of a sweat amount of the user, a physical activity level of the user, a sleeping time and habit of the user, or a heart rate of the user.

21. A method of measuring a blood glucose level, the method comprising:
transmitting, via an oscillator assembly, microwaves at an oscillator frequency based on an input impedance, wherein the input impedance is associated with the permittivity of blood in a user's blood vessel;
detecting, via a frequency detection circuit, a first oscillator frequency at a first time;
detecting, via the frequency detection circuit, a second oscillator frequency at a second time;
receiving, via a main control board, an indication of a condition of the user;
comparing, via the main control board, the first oscillator frequency with the second oscillator frequency to determine a frequency drift;
calibrating, via the main control board, the frequency drift based on the received indication of the condition of the user; and
determining, via the main control board, a blood glucose level of the user based on the calibrated frequency drift.

22. The method of claim 21, wherein transmitting microwaves at the oscillator frequency comprises continuously transmitting microwaves at the oscillator frequency.

23. The method of claim 21, wherein the oscillator frequency that is detected is in a range from 1 gigahertz to 10 gigahertz.

24. The method of claim 21, wherein the oscillator frequency that is detected is in a range from 4 gigahertz to 8 gigahertz.

25. The method of claim 21 further comprising providing an output via a user output component, wherein the output is indicative of the determined blood glucose level.

26. The method of claim 21, wherein the condition of the user that is detected includes at least one of an ambient temperature, a sweat amount of the user, a physical activity level of the user, a sleeping time and habit of the user, or a heart rate of the user.
